(19) United States
(12) Patent Application Publication
(10) Pub. No.: US 2025/0302797 A1
(43) Pub. Date: Oct. 2, 2025

(54) COMBINATION THERAPIES FOR TREATING INFLAMMATION

(71) Applicant: Flagship Pioneering Innovations VI, LLC, Cambridge, MA (US)

(72) Inventors: Michael Ka Chun Wong, Arlington, MA (US); Hok Hei Tam, Newton, MA (US); David Barry Kolesky, Arlington, MA (US); Fernanda Menezes Cerqueira, Winthrop, MA (US)

(21) Appl. No.: 19/101,989

(22) PCT Filed: Aug. 10, 2023
(86) PCT No.: PCT/US2023/029958

§ 371 Date: Feb. 7, 2025

Related U.S. Application Data
(60) Provisional application No. 63/396,936, filed on Aug. 10, 2022.

(51) Int. Cl. A61K 31/352; A61P 17/00; A61P 29/00

(52) U.S. Cl. CPC A61K 31/352; A61P 17/00; A61P 29/00

(57) ABSTRACT

The present disclosure relates to therapeutic agents and combinations thereof (e.g., pharmaceutical compositions) for the treatment of inflammation and an inflammatory disease or disorder in a subject, tissue or cell.

### CLAIM OF PRIORITY

The present application claims priority to U.S. Provisional Application No. 63/396,936, filed Aug. 10, 2022; the contents of which are incorporated herein by reference in their entirety.

### BACKGROUND

There is a need for therapeutic agents useful in modulating the inflammatory response in a cell or tissue, and treatment of inflammation and inflammatory diseases and disorders in a subject.

### SUMMARY

The present disclosure relates to therapeutic agents and combinations thereof (e.g., pharmaceutical compositions) for the treatment of inflammation and inflammatory diseases and disorders in a subject, tissue or cell. In one aspect, the present disclosure features a method of treating inflammation or an inflammatory disease or disorder (e.g., a dermatological condition) in a subject comprising administering a combination of a flavonoid (e.g., luteolin) and a xanthone (e.g., gamma-mangostin) to the subject. In an embodiment, administering a combination of a flavonoid (e.g., luteolin) and a xanthone (e.g., gamma-mangostin) to a subject results in a beneficial effect in the subject, e.g., compared with administering the flavonoid and/or xanthone individually. For example, administering a combination of a flavonoid and a xanthone to a cell or subject may result in one or more of: (i) reducing the level of an inflammatory cytokine (e.g., IL-6, IL-1b, and TNF-a) in a cell or subject; (ii) increasing the level of an anti-inflammatory cytokine (e.g., IL-4 and IL-10) in a cell or subject; (iii) reducing the level of a hypersensitivity marker (e.g., IL-13, IL-14, INF-g, and serum IgE) in a cell or subject; (iv) inducing expression of an anti-inflammatory macrophage (e.g., M2 macrophage) in a cell or subject; (v) improving skin health in a cell or subject; (vi) improving a symptom of a dermatological condition (e.g., atopic dermatitis, psoriasis, and dermal hypersensitivity) in a cell or subject; and (vii) improving a symptom of inflammation or an inflammatory disease or disorder in a cell or subject.

### BRIEF DESCRIPTION OF DRAWINGS

FIGS. 1A-1D are graphs showing the levels of secreted cytokines from primary human T cells treated with IL-2 (positive control) or combination of luteolin and gamma-mangostin.

FIG. 2 is a graph depicting ear thickness measurements of oxazolone-stimulated ears or unstimulated control ears after topical treatment with either vehicle, dexamethasone (control), or luteolin+gamma-mangostin.

FIGS. 3A-3C are graphs showing the levels of certain cytokines (FIG. 3A—MIP1-beta, FIG. 3B—CXCL1, FIG. 3C—TNFa) in mice pre-injected for 1 hour with vehicle, dexamethasone (control), or luteolin+gamma-mangostin, then injected with LPS.

### DESCRIPTION

Described herein are compositions comprising (i) a flavonoid or a pharmaceutically acceptable salt thereof and (ii) a xanthone or a pharmaceutically acceptable salt thereof, as well as compositions and methods of using the same. In an embodiment, administering a combination of a flavonoid and a xanthone to a subject results in a beneficial effect in the subject, e.g., compared with administering the flavonoid and/or the xanthone individually. For example, a combination of a flavonoid and a xanthone may result reducing the level of an inflammatory cytokine in a cell or subject, or treatment of a dermatological condition in a cell or subject. A description of exemplary embodiments of the disclosure is provided herein.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of a cell titer or a bodily fluid, e.g., via mass spectroscopy (e.g. LC-MS) or PCR (e.g., RT-PCR).

“Co-administration”, “co-administering”, “co-providing”, “in combination” and “a combination of” as used herein in the context of the administration of a flavonoid and a xanthone, refers to administration at the same time or administration of one therapy before (e.g., immediately before, less than about 5, about 10, about 15, about 30, about 45, about 60 minutes, about 1, about 2, about 3, about 4, about 6, about 8, about 10, about 12, about 16, about 20, about 24, about 48, about 72 or more hours before) administration of a secondary therapy. In some embodiments, the therapies to be co-administered are formulated in a single composition. In other embodiments, the therapies to be co-administered are formulated separately.

Numerous ranges, e.g., ranges for the amount of a therapy administered per day, are provided herein. In some embodiments, the range includes both endpoints. In other embodiments, the range excludes one or both endpoints. By way of example, the range can exclude the lower endpoint. Thus, in such an embodiment, a range of 250 to 400 mg/day, excluding the lower endpoint, would cover an amount greater than 250 that is less than or equal to 400 mg/day.

The term “comprise” is intended to mean “include”. Where a term is provided in the singular, it also contemplates aspects of the invention described by the plural of that term. The term “and/or” where used herein is to betaken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

A “course” or “course of therapy,” as referred to herein, comprises one or more separate administrations of a therapeutic agent or a combination of therapeutic agents (e.g., a flavonoid and/or a xanthone). A course of therapy can comprise one or more cycles of a therapeutic agent. In some embodiments, a therapeutic agent is administered to a subject at least once, at least twice, at least three times, at least four times, or more over a course of treatment. A subject may be administered with one or more courses of treatment. In some embodiments, rest periods may be interposed between courses of treatment. For example, a rest period may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

A“cycle”, as used herein in the context of a cycle of administration of a therapeutic agent or a combination of therapeutic agents, refers to a period of time for which the therapeutic agent or combination of therapeutic agents is administered to a patient. For example, if a therapeutic agent is administered for a cycle of 4 weeks days, the periodic administration, e.g., daily or twice daily, is given for 4 weeks. A therapeutic agent or combination of therapeutic agents can be administered for more than one cycle. In some embodiments, the first and second or subsequent cycles are the same in terms of one or both of duration and periodic administration. In embodiments, a first and second or subsequent cycle differs in terms of one or both of duration and periodic administration. Rest periods may be interposed between cycles. A rest cycle may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

The term “efficacy,” as used herein in the context of a therapeutic agent or combination of therapeutic agents, refers to the ability of a therapeutic agent or a combination of therapeutic agents to effect a desirable treatment outcome, such as (i) reducing the level of an inflammatory cytokine (e.g., IL-6, IL-1b, and TNF-a) in a cell or subject; (ii) increasing the level of an anti-inflammatory cytokine (e.g., IL-4 and TL-10) in a cell or subject; (iii) reducing the level of a hypersensitivity marker (e.g., IL-13, IL-14, INF-g, and serum IgE) in a cell or subject; (iv) inducing expression of an anti-inflammatory macrophage (e.g., M2 macrophage) in a cell or subject; (v) improving skin health in a cell or subject; (vi) improving a symptom of a dermatological condition (e.g., atopic dermatitis, psoriasis, and dermal hypersensitivity) in a cell or subject; and (vii) improving a symptom of inflammation or an inflammatory disease or disorder in a cell or subject.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a combination of a flavonoid and a xanthone described herein, the amount of a marker of a metric (e.g., cell viability, level of a cytokine) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

As used herein, the terms “prevent” or “preventing” as used in the context of a disease or disorder described herein (e.g., inflammation or an inflammatory disease or disorder), refer to administration of a flavonoid in combination with a xanthone to a subject, e.g., the administration of a gamma-mangostin and luteolin, such that the onset of at least one symptom of the disorder or disease is delayed as compared to what would be seen in the absence of administration of said combination.

As used herein, the term “subject” is intended to include human and non-human animals. Exemplary human subjects include a human patient having a disease or disorder, e.g., a disorder described herein (e.g., inflammation or an inflammatory disease or disorder), or a healthy subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, an amount of a compound, conjugate, or substance effective to treat a disease or disorder (e.g., inflammation or an inflammatory disease or disorder), “therapeutically effective amount,” “effective amount” or “effective course” refers to an amount of the compound or composition which is effective, upon single or multiple dose administration(s) to a subject, in treating a subject, or in curing, alleviating, relieving or improving a subject with inflammation or an inflammatory disease or disorder beyond that expected in the absence of such treatment.

As used herein, the terms “treat” or “treating” as used in the context of a disease or disorder described herein (e.g., inflammation or an inflammatory disease or disorder), refer to administration of a flavonoid in combination with a xanthone to a subject, e.g., the administration of gamma-mangostin and luteolin, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease. In some embodiments, treating includes preventing. In some embodiments, treating does not include preventing.

### Therapeutic Agents

Described herein are flavonoids administered in combination with xanthones to provide a therapeutic benefit to a cell or subject, e.g., treating inflammation or an inflammatory disease or disorder.

### Flavonoids

Flavonoids are class of polyphenolic compounds containing at least one heterocyclic ring comprising a heteroatom, such as an oxygen atom. Flavonoids may be derived from plants, where they carry out multiple functions. For example, flavonoids play a role in plant pigmentation, UV filtration, nitrogen fixation, and certain metabolic pathways. Depending on their structure, flavonoids may be further classified into subgroups such as anthocyanidins, chalcones, flavonols, flavanones, and isoflavonoids.

In an embodiment, the flavonoid is a compound of Formula (A):

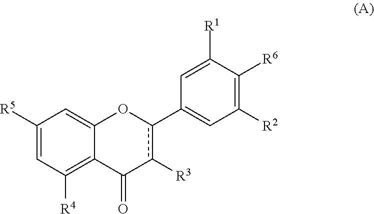

or a pharmaceutically acceptable salt thereof, wherein each of R¹, R², R³, R⁴, R⁵, and R⁶ is independently hydrogen or —OR^{A}; R^{A} is hydrogen, C₁-C₆ alkyl, or cycloalkyl; and “ [symbol] ” is a single or double bond.

[symbol]

In an embodiment, R¹ is —OR^{A}. In an embodiment, R¹ is —OH. In an embodiment, R² is -hydrogen. In an embodiment, R³ is hydrogen. In an embodiment, R⁴ is —OR^{A}. In an embodiment, R⁴ is —OH. In an embodiment, [symbol] is a double bond.

In an embodiment, the flavonoid is a compound of Formula (A-i):

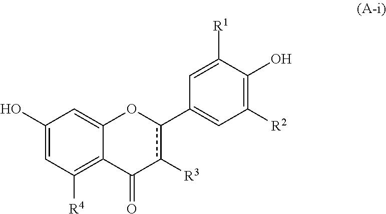

or a pharmaceutically acceptable salt thereof, wherein each of R¹ and R² is independently hydrogen or —OR^{A}; R³ is hydrogen or —OR^{A}; R⁴ is hydrogen or —OR^{A}; R^{A} is hydrogen, C₁-C₆ alkyl, or cycloalkyl; and “ [symbol] ” is a single or double bond.

In an embodiment, R¹ is —OR^{A}. In an embodiment, R¹ is —OH. In an embodiment, R² is -hydrogen. In an embodiment, R³ is hydrogen. In an embodiment, R⁴ is —OR^{A}. In an embodiment, R⁴ is —OH. In an embodiment, [symbol] is a double bond.

In an embodiment, the flavonoid is a compound of Formula (A-ii):

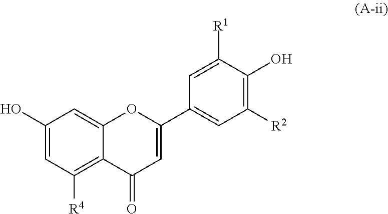

or a pharmaceutically acceptable salt thereof, wherein each of R¹ and R² is independently hydrogen or —OR^{A}; R⁴ is hydrogen or —OR^{A}; and R^{A} is hydrogen, C₁-C₆ alkyl, or cycloalkyl.

In an embodiment of Formula (A-i), R¹ is —OR^{A}; R² is hydrogen; and R⁴ is —OR^{A}. In an embodiment of Formula (A-i), R¹ is —OH; R² is hydrogen; and R⁴ is —OH.

In an embodiment, the flavonoid is a compound of Formula (A-iii):

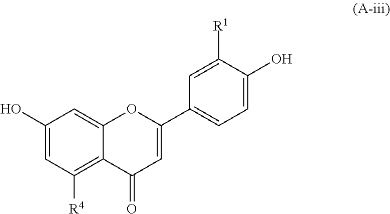

or a pharmaceutically acceptable salt thereof, wherein each of R¹ is hydrogen or —OR^{A}; R⁴ is hydrogen or —OR^{A}; and R^{A} is hydrogen, C₁-C₆ alkyl, or cycloalkyl.

In an embodiment of Formula (A-iii), R¹ is —OR^{A} and R⁴ is —OR^{A}. In an embodiment of Formula (A-ii), R¹ is —OH and R⁴ is —OH.

In an embodiment, the flavonoid is a compound shown in Table 1.

TABLE 1
Exemplary flavonoids

| Compound | Structure |
|---|---|
| Luteolin | 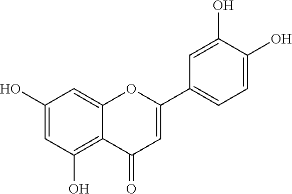 [SMILES: O=c1cc(-c2ccc(O)c(O)c2)oc2cc(O)cc(O)c12] |
| Quercetin | 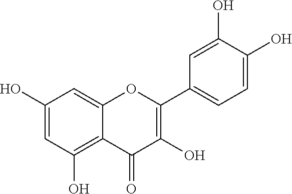 [SMILES: O=c1c(O)c(-c2ccc(O)c(O)c2)oc2cc(O)cc(O)c12] |
| Myricetin | 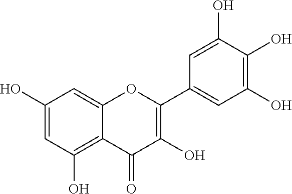 [SMILES: O=c1c(O)c(-c2cc(O)c(O)c(O)c2)oc2cc(O)cc(O)c12] |
| Taxifolin | 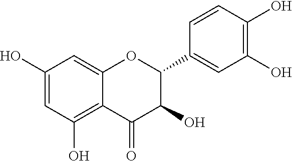 [SMILES: O=C1c2c(O)cc(O)cc2O[C@H](c2ccc(O)c(O)c2)[C@H]1O] |
| Fisetin | 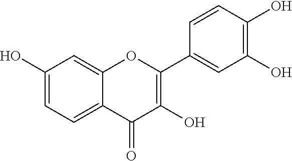 [SMILES: O=c1c(O)c(-c2ccc(O)c(O)c2)oc2cc(O)ccc12] |
| Kaempferol | 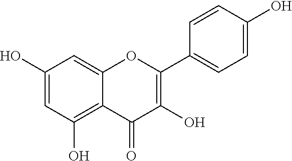 [SMILES: O=c1c(O)c(-c2ccc(O)cc2)oc2cc(O)cc(O)c12] |
| Apigenin | 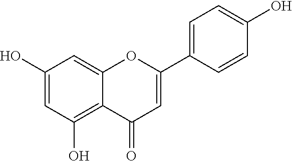 [SMILES: O=c1cc(-c2ccc(O)cc2)oc2cc(O)cc(O)c12] |
| Tricetin | 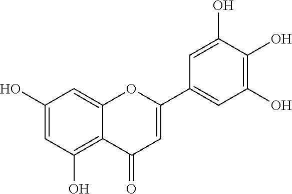 [SMILES: O=c1cc(-c2cc(O)c(O)c(O)c2)oc2cc(O)cc(O)c12] |
| Isohamnetin | 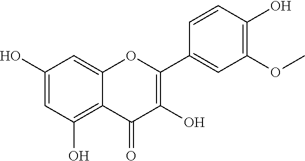 [SMILES: COc1cc(-c2oc3cc(O)cc(O)c3c(=O)c2O)ccc1O] |
| Eriodictyol | 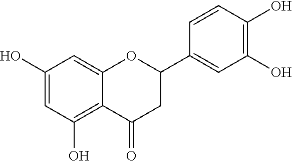 [SMILES: O=C1CC(c2ccc(O)c(O)c2)Oc2cc(O)cc(O)c21] |
| Dihydromyricetin | 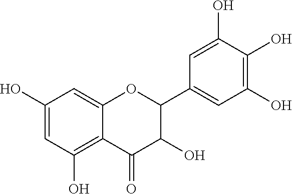 [SMILES: O=C1c2c(O)cc(O)cc2OC(c2cc(O)c(O)c(O)c2)C1O] |
| Chryseriol | 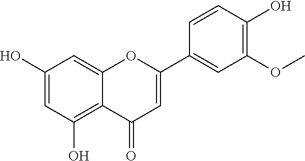 [SMILES: COc1cc(-c2cc(=O)c3c(O)cc(O)cc3o2)ccc1O] |
| 7,3 [symbol] , 4 [symbol] Trihydroxyflavone | 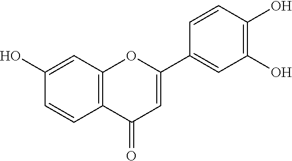 [SMILES: O=c1cc(-c2ccc(O)c(O)c2)oc2cc(O)ccc12] |
| 4,7- Dihydroxyflavone | 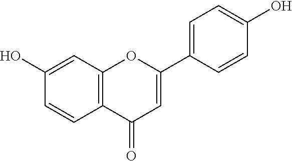 [SMILES: O=c1cc(-c2ccc(O)cc2)oc2cc(O)ccc12] |
| Hesperitin | 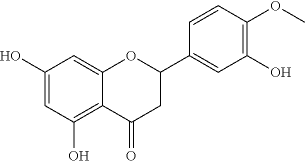 [SMILES: COc1ccc(C2CC(=O)c3c(O)cc(O)cc3O2)cc1O] |
| Galangin | 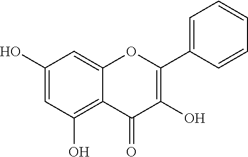 [SMILES: O=c1c(O)c(-c2ccccc2)oc2cc(O)cc(O)c12] |
| Chrysin | 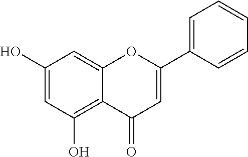 [SMILES: O=c1cc(-c2ccccc2)oc2cc(O)cc(O)c12] |
| 7-O- Methylluteolin | 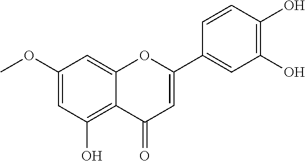 [SMILES: COc1cc(O)c2c(=O)cc(-c3ccc(O)c(O)c3)oc2c1] |
| Genkwanin | 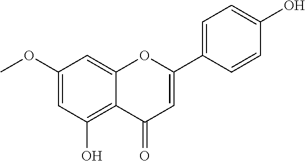 [SMILES: COc1cc(O)c2c(=O)cc(-c3ccc(O)cc3)oc2c1] |
| Acacetin | 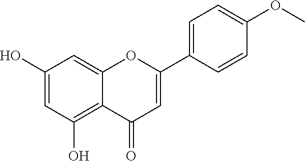 [SMILES: COc1ccc(-c2cc(=O)c3c(O)cc(O)cc3o2)cc1] |
| Restusin | 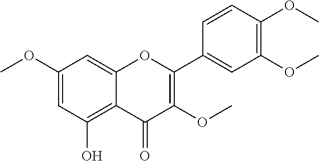 |
| 7-Hydroxyflavone | 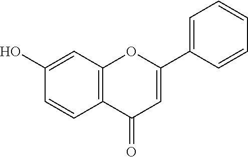 [SMILES: O=c1cc(-c2ccccc2)oc2cc(O)ccc12] |
| Apigenin 7,4- dimethyl ether | 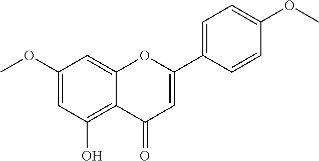 |
| Pratol | 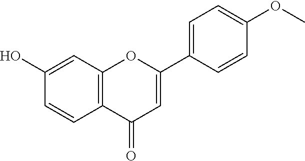 [SMILES: COc1ccc(-c2cc(=O)c3ccc(O)cc3o2)cc1] |
| Ombuin | 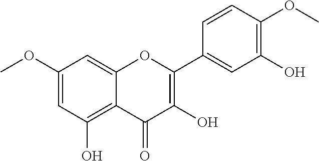 |
| Quercetin 3,3- dimethyl ether | 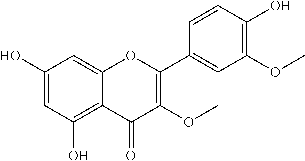 [SMILES: COc1cc(-c2oc3cc(O)cc(O)c3c(=O)c2OC)ccc1O] |
| Tectochrysin | 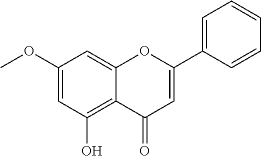 [SMILES: COc1cc(O)c2c(=O)cc(-c3ccccc3)oc2c1] |
| Tricin | 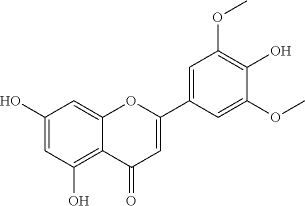 [SMILES: COc1cc(-c2cc(=O)c3c(O)cc(O)cc3o2)cc(OC)c1O] |

[symbol]

indicates data missing or illegible when filed

In an embodiment, the flavonoid is luteolin or a pharmaceutically acceptable salt thereof. In an embodiment, the flavonoid is quercetin or a pharmaceutically acceptable salt thereof. In an embodiment, the flavonoid is myricetin or a pharmaceutically acceptable salt thereof. In an embodiment, the flavonoid is taxifolin or a pharmaceutically acceptable salt thereof. In an embodiment, the flavonoid is fisetin or a pharmaceutically acceptable salt thereof. In an embodiment, the flavonoid is kaempferol or a pharmaceutically acceptable salt thereof.

In an embodiment, the flavonoid is apigenin or a pharmaceutically acceptable salt thereof. In an embodiment, the flavonoid is tricetin or a pharmaceutically acceptable salt thereof. In an embodiment, the flavonoid is isohamnetin or a pharmaceutically acceptable salt thereof. In an embodiment, the flavonoid is eriodictyol or a pharmaceutically acceptable salt thereof. In an embodiment, the flavonoid is dihydromyricetin or a pharmaceutically acceptable salt thereof. In an embodiment, the flavonoid is chryseriol or a pharmaceutically acceptable salt thereof. In an embodiment, the flavonoid is 7,3,4-trihydroxyflavone or a pharmaceutically acceptable salt thereof. In an embodiment, the flavonoid is 4,7-dihydroxyflavone or a pharmaceutically acceptable salt thereof. In an embodiment, the flavonoid is hesperitin or a pharmaceutically acceptable salt thereof. In an embodiment, the flavonoid is galangin or a pharmaceutically acceptable salt thereof. In an embodiment, the flavonoid is chrysin or a pharmaceutically acceptable salt thereof. In an embodiment, the flavonoid is 7-O-methylluteolin or a pharmaceutically acceptable salt thereof. In an embodiment, the flavonoid is genkwanin or a pharmaceutically acceptable salt thereof. In an embodiment, the flavonoid is acacetin or a pharmaceutically acceptable salt thereof. In an embodiment, the flavonoid is retusin or a pharmaceutically acceptable salt thereof. In an embodiment, the flavonoid is 7-hydroxyflavone or a pharmaceutically acceptable salt thereof. In an embodiment, the flavonoid is apigenin 7,4-dimethyl ether or a pharmaceutically acceptable salt thereof. In an embodiment, the flavonoid is Pratol or a pharmaceutically acceptable salt thereof. In an embodiment, the flavonoid is ombuin or a pharmaceutically acceptable salt thereof. In an embodiment, the flavonoid is quercetin 3,3-dimethyl ether or a pharmaceutically acceptable salt thereof. In an embodiment, the flavonoid is tectochrysin or a pharmaceutically acceptable salt thereof.

In an embodiment, the flavonoid is luteolin or a pharmaceutically acceptable salt thereof, provided substantially free of another flavonoid such as quercetin, myricetin, taxifolin, fistein, or kaempferol. In an embodiment, the flavonoid is quercetin or a pharmaceutically acceptable salt thereof, provided substantially free of another flavonoid such as luteolin, myricetin, taxifolin, fistein, or kaempferol. In an embodiment, the flavonoid is myricetin or a pharmaceutically acceptable salt thereof, provided substantially free of another flavonoid such as quercetin, luteolin, taxifolin, fistein, or kaempferol. In an embodiment, the flavonoid is taxifolin or a pharmaceutically acceptable salt thereof, provided substantially free of another flavonoid such as quercetin, myricetin, luteolin, fistein, or kaempferol. In an embodiment, the flavonoid is fisetin or a pharmaceutically acceptable salt thereof, provided substantially free of another flavonoid such as quercetin, myricetin, taxifolin, luteolin, or kaempferol. In an embodiment, the flavonoid is kaempferol or a pharmaceutically acceptable salt thereof, provided substantially free of another flavonoid such as quercetin, myricetin, taxifolin, fistein, or luteolin.

In an embodiment, the flavonoid is luteolin or a pharmaceutically acceptable salt thereof, provided substantially free of another flavonoid shown in Table 1. In an embodiment, the flavonoid is quercetin or a pharmaceutically acceptable salt thereof, provided substantially free of another flavonoid shown in Table 1. In an embodiment, the flavonoid is myricetin or a pharmaceutically acceptable salt thereof, provided substantially free of another flavonoid shown in Table 1. In an embodiment, the flavonoid is taxifolin or a pharmaceutically acceptable salt thereof, provided substantially free of another flavonoid s shown in Table 1. In an embodiment, the flavonoid is fisetin or a pharmaceutically acceptable salt thereof, provided substantially free of another flavonoid shown in Table 1. In an embodiment, the flavonoid is kaempferol or a pharmaceutically acceptable salt thereof, provided substantially free of another flavonoid s shown in Table 1.

In an embodiment, the flavonoid or a pharmaceutically acceptable salt thereof is provided as a substantially pure compound, for example, at least 85%, at least 90%, at least 95%, at least 98%, at least 99%, or at least 99.9% pure, measured, e.g, by HPLC analysis. In an embodiment, the flavonoid is luteolin or a pharmaceutically acceptable salt thereof and is provided as a substantially pure compound, for example, at least 85%, at least 90%, at least 95%, at least 98%, at least 99%, or at least 99.9% pure, measured, e.g, by HPLC analysis.

In an embodiment, the flavonoid is provided with less than 10%, 7.5%, 5%, 2.5%, 1%, 0.5%, or 0.1% of another flavonoid, e.g., in the absence of another flavonoid. In an embodiment, the flavonoid is luteolin and is provided with less than 10%, 7.5%, 5%, 2.5%, 1%, 0.5%, or 0.1% of another flavonoid, e.g., in the absence of another flavonoid. In an embodiment, the flavonoid is luteolin and is provided with less than 10%, 7.5%, 5%, 2.5%, 1%, 0.5%, or 0.1% of quercetin present, e.g., in the absence of quercetin. In an embodiment, the flavonoid is provided with less than 10%, 7.5%, 5%, 2.5%, 1%, 0.5%, or 0.1% of another plant-derived substance, such as a plant metabolite, plant lipid, or plant fiber. In an embodiment, the flavonoid is luteolin and is provided with less than 10%, 7.5%, 5%, 2.5%, 1%, 0.5%, or 0.10% of plant-derived substance, such as a plant metabolite, plant lipid, or plant fiber.

A composition useful for the treatment of inflammation or an inflammatory disease or disorder may contain a flavonoid (e.g., luteolin) or a plurality of flavonoids. For example, a composition comprising a flavonoid may comprise luteolin, e.g., synthetically prepared or extracted from a natural source, in the absence of another flavonoid. In contrast, a composition comprising a flavonoid may also contain a combination of luteolin and a closely related analog or variant thereof, e.g., a flavonoid shown in Table 1, e.g., quercetin.

### Xanthones

Xanthones are class of tricyclic polyphenol compounds commonly found in higher plants, fungi, and lichens. This class of compounds often contains a glycoside or short lipid tail appended to the tricyclic core, and related analogs thereof. Xanthones are involved in multiple pathways within the plant, including UV filtration and cell signaling.

In an embodiment, the xanthone is a compound of Formula (B):

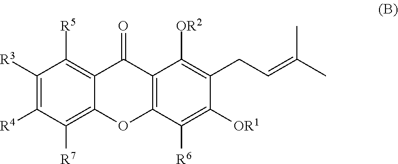

or a pharmaceutically acceptable salt thereof, wherein each of R¹ and R² is independently hydrogen or C₁-C₆ alkyl; each of R³, R⁴, and R⁷ is independently hydrogen, —OR^{A}, C₁-C₆ alkyl, C₂-C₆ alkenyl, C₂-C₆ alkynyl, or C₂-C₁₀ alkenyl; each of R⁵ and R⁶ is independently hydrogen, C₁-C₆ alkyl, C₂-C₆ alkenyl, C₂-C₆ alkynyl, —OR^{A} or C₂-C₁₀ alkenyl; and R^{A} is hydrogen, C₁-C₆ alkyl, or cycloalkyl.

In an embodiment, the xanthone is a compound of Formula (B-i):

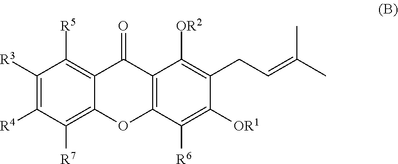

or a pharmaceutically acceptable salt thereof, wherein each of R¹ and R² is independently hydrogen or C₁-C₆ alkyl; each of R³ and R⁴ is independently hydrogen or —OR^{A}; each of R⁵ and R⁶ is independently hydrogen, C₁-C₆ alkyl, C₂-C₆ alkenyl, C₂-C₆ alkynyl, or —OR^{A}; and R^{A} is hydrogen, C₁-C₆ alkyl, or cycloalkyl.

In an embodiment, R¹ is hydrogen. In an embodiment, R² is hydrogen. In an embodiment, R is —OR^{A}. In an embodiment, R³ is —OH. In an embodiment, R⁴ is —OR^{A}. In an embodiment, R⁴ is —OH. In an embodiment, R⁵ is C₂-C₆ alkenyl. In an embodiment, R⁵ is —CH₂CH═C(CH₃)₂. In an embodiment, R⁶ is hydrogen.

In an embodiment of Formula (B-i), each of R¹ and R² is hydrogen; each of R³ and R⁴ is —OR^{A}; R⁵ is C₂-C₆ alkenyl and R⁶ is hydrogen.

In an embodiment, the xanthone is a compound of Formula (B-ii):

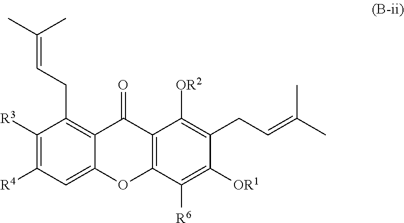

or a pharmaceutically acceptable salt thereof, wherein each of R¹ and R² is independently hydrogen or C₁-C₆ alkyl; each of R³ and R⁴ is independently hydrogen or —OR^{A}; R^{A} is hydrogen, C₁-C₆ alkyl, C₂-C₆ alkenyl, C₂-C₆ alkynyl, or —OR^{A}; and R^{A} is hydrogen, C₁-C₆ alkyl, or cycloalkyl.

In an embodiment of Formula (B-ii), each of R¹ and R² is hydrogen; each of R³ and R⁴ is —OR^{A}; and R⁶ is hydrogen.

In an embodiment, the xanthone is a compound of Formula (B-iii):

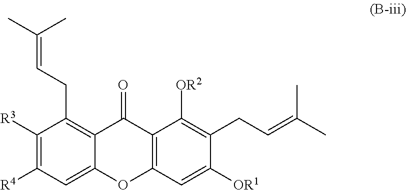

or a pharmaceutically acceptable salt thereof, wherein each of R¹ and R² is independently hydrogen or C₁-C₆ alkyl; each of R³ and R⁴ is independently hydrogen or —OR^{A}; and R^{A} is hydrogen, C₁-C₆ alkyl, or cycloalkyl.

In an embodiment of Formula (B-ii), each of R¹ and R² is hydrogen; and each of R³ and R⁴ is —OR^{A}.

In an embodiment of Formula (B-ii), each of R¹ and R² is hydrogen; R³ is —OCH₃, and R⁴ is —OH.

In an embodiment, the xanthone is a compound shown in Table 2.

TABLE 2
Exemplary xanthones

| Compound | Structure |
|---|---|
| Alpha-Mangostin | 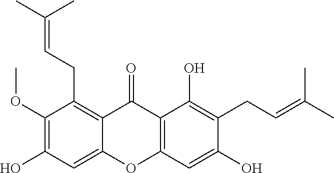 [SMILES: COc1c(O)cc2oc3cc(O)c(CC=C(C)C)c(O)c3c(=O)c2c1CC=C(C)C] |
| Beta-Mangostin | 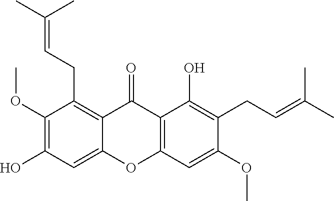 [SMILES: COc1cc2oc3cc(O)c(OC)c(CC=C(C)C)c3c(=O)c2c(O)c1CC=C(C)C] |
| Gamma-Mangostin | 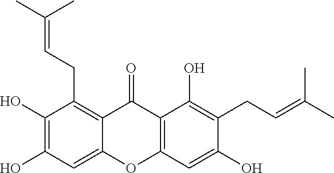 [SMILES: CC(C)=CCc1c(O)cc2oc3cc(O)c(O)c(CC=C(C)C)c3c(=O)c2c1O] |
| 8-deoxygartanin | 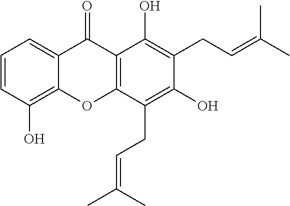 [SMILES: CC(C)=CCc1c(O)c(CC=C(C)C)c2oc3c(O)cccc3c(=O)c2c1O] |
| Garcinone E | 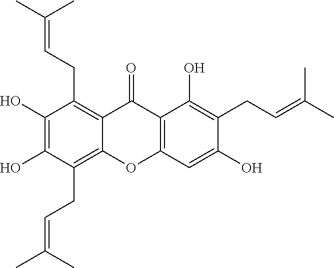 [SMILES: CC(C)=CCc1c(O)cc2oc3c(CC=C(C)C)c(O)c(O)c(CC=C(C)C)c3c(=O)c2c1O] |
| Gartanin | 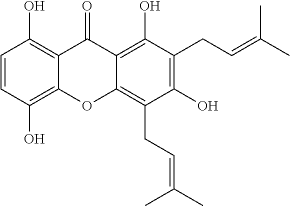 [SMILES: CC(C)=CCc1c(O)c(CC=C(C)C)c2oc3c(O)ccc(O)c3c(=O)c2c1O] |
| 1,7-dihydroxy-3- methoxy-2- prenylxanthone | 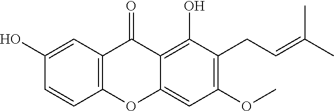 [SMILES: COc1cc2oc3ccc(O)cc3c(=O)c2c(O)c1CC=C(C)C] |
| 1,7-dihydroxy-3,6- dimethoxy-2,8- diprenylxanthone | 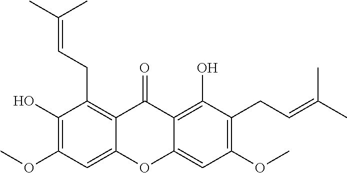 [SMILES: COc1cc2oc3cc(OC)c(CC=C(C)C)c(O)c3c(=O)c2c(CC=C(C)C)c1O] |
| Morusignin B | 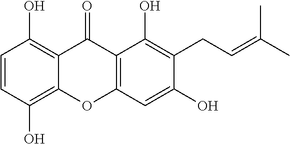 [SMILES: CC(C)=CCc1c(O)cc2oc3c(O)ccc(O)c3c(=O)c2c1O] |
| Dulxanthone B | 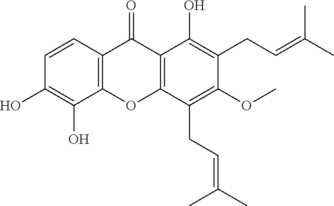 [SMILES: COc1c(CC=C(C)C)c(O)c2c(=O)c3ccc(O)c(O)c3oc2c1CC=C(C)C] |
| 1,5-dihydroxy-3- methoxy-2- prenylxanthone | 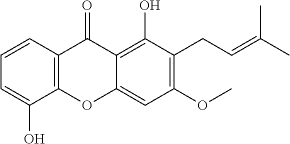 [SMILES: COc1cc2oc3c(O)cccc3c(=O)c2c(O)c1CC=C(C)C] |
| 1,4,6-trihydroxy-5- methoxy-7- prenylxanthone | 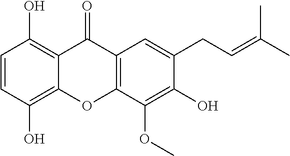 [SMILES: COc1c(O)c(CC=C(C)C)cc2c(=O)c3c(O)ccc(O)c3oc12] |
| Nigrolineaxanthone E | 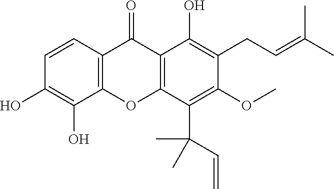 [SMILES: C=CC(C)(C)c1c(OC)c(CC=C(C)C)c(O)c2c(=O)c3ccc(O)c(O)c3oc12] |
| 1-Hydroxy-3,5- dimethoxy-2,4- diprenylxanthone | 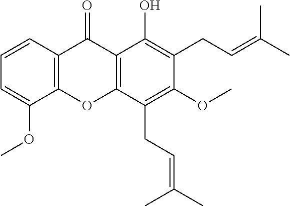 [SMILES: COc1c(CC=C(C)C)c(O)c2c(=O)c3cccc(OC)c3oc2c1CC=C(C)C] |
| Gerontoxanthone 1 | 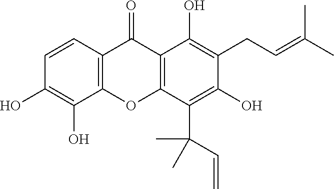 [SMILES: C=CC(C)(C)c1c(O)c(CC=C(C)C)c(O)c2c(=O)c3ccc(O)c(O)c3oc12] |
| 6-deoxy-gamma- mangostin | 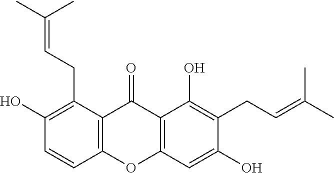 [SMILES: CC(C)=CCc1c(O)cc2oc3ccc(O)c(CC=C(C)C)c3c(=O)c2c1O] |
| Cowanin | 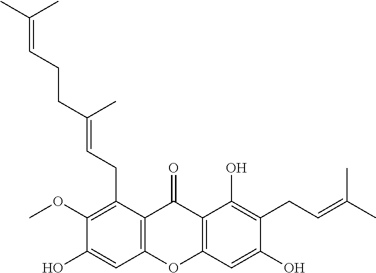 [SMILES: COc1c(O)cc2oc3cc(O)c(CC=C(C)C)c(O)c3c(=O)c2c1C/C=C(\C)CCC=C(C)C] |
| Dulcisxanthone B | 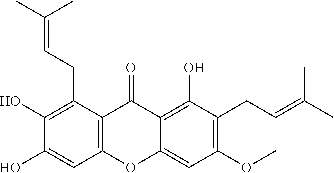 [SMILES: COc1cc2oc3cc(O)c(O)c(CC=C(C)C)c3c(=O)c2c(O)c1CC=C(C)C] |
| Cowaxanthone B | 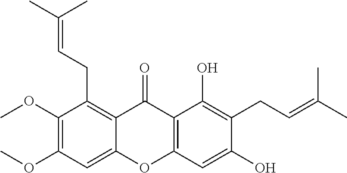 [SMILES: COc1cc2oc3cc(O)c(CC=C(C)C)c(O)c3c(=O)c2c(CC=C(C)C)c1OC] |
| 1,3,7-trihydroxy- 2,4- diisoprenylxanthone | 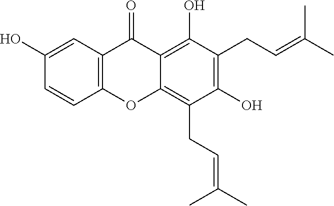 [SMILES: CC(C)=CCc1c(O)c(CC=C(C)C)c2oc3ccc(O)cc3c(=O)c2c1O] |
| Cratoxyarborenone B | 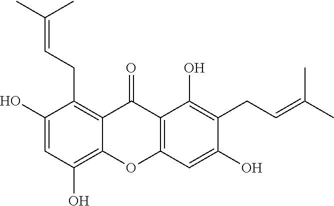 [SMILES: CC(C)=CCc1c(O)cc2oc3c(O)cc(O)c(CC=C(C)C)c3c(=O)c2c1O] |
| 1,3,5-trihydroxy-2- prenyl-9H- xanthene-9-one | 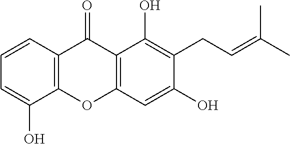 [SMILES: CC(C)=CCc1c(O)cc2oc3c(O)cccc3c(=O)c2c1O] |
| 1,3,7-trihydroxy-2- prenylxanthone | 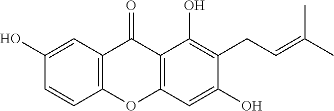 [SMILES: CC(C)=CCc1c(O)cc2oc3ccc(O)cc3c(=O)c2c1O] |
| Doitunggarcinone C | 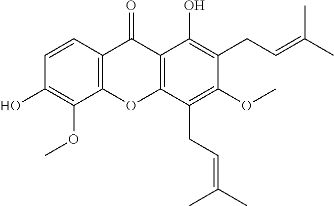 [SMILES: COc1c(CC=C(C)C)c(O)c2c(=O)c3ccc(O)c(OC)c3oc2c1CC=C(C)C] |
| Cudratricusxanthone K | 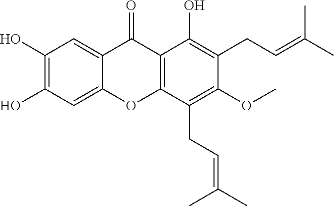 [SMILES: COc1c(CC=C(C)C)c(O)c2c(=O)c3cc(O)c(O)cc3oc2c1CC=C(C)C] |
| 1-hydroxy-3,5- dimethoxy-2- prenylxanthone | 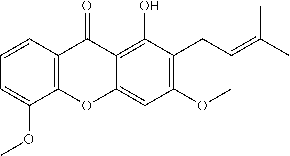 [SMILES: COc1cc2oc3c(OC)cccc3c(=O)c2c(O)c1CC=C(C)C] |
| 1,8-dihydroxy-3,5- dimethoxy-2- prenylxanthone | 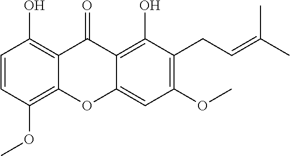 [SMILES: COc1cc2oc3c(OC)ccc(O)c3c(=O)c2c(O)c1CC=C(C)C] |

In an embodiment, the xanthone is alpha-mangostin or a pharmaceutically acceptable salt thereof. In an embodiment, the xanthone is beta-mangostin or a pharmaceutically acceptable salt thereof. In an embodiment, the xanthone is gamma-mangostin or a pharmaceutically acceptable salt thereof. In an embodiment, the xanthone is 8-deoxygartanin or a pharmaceutically acceptable salt thereof.

In an embodiment, the xanthone is garcinone E or a pharmaceutically acceptable salt thereof. In an embodiment, the xanthone is gartanin or a pharmaceutically acceptable salt thereof. In an embodiment, the xanthone is 1,7-dihydroxy-3-methoxy-2-prenylxanthone or a pharmaceutically acceptable salt thereof. In an embodiment, the xanthone is 1,7-dihydroxy-3,6-dimethoxy-2,8-diprenylxanthone or a pharmaceutically acceptable salt thereof. In an embodiment, the xanthone is morusignin B or a pharmaceutically acceptable salt thereof. In an embodiment, the xanthone is dulxanthone B or a pharmaceutically acceptable salt thereof in an embodiment, the xanthone is 1,5-dihydroxy-3-methoxy-2-prenylxanthone or a pharmaceutically acceptable salt thereof. In an embodiment, the xanthone is 1,4,6-trihydroxy-5-methoxy-7-prenylxanthone or a pharmaceutically acceptable salt thereof. In an embodiment, the xanthone is nigrolineaxanthone E or a pharmaceutically acceptable salt thereof. In an embodiment, the xanthone is 1-hydroxy-3,5-dimethoxy-2,4-diprenylxanthone or a pharmaceutically acceptable salt thereof. In an embodiment, the xanthone is gerontoxanthone I or a pharmaceutically acceptable salt thereof. In an embodiment, the xanthone is 6-deoxy-gamma-mangostin or a pharmaceutically acceptable salt thereof. In an embodiment, the xanthone is cowanin or a pharmaceutically acceptable salt thereof. In an embodiment, the xanthone is dulcisxanthone B or a pharmaceutically acceptable salt thereof. In an embodiment, the xanthone is cowaxanthone B or a pharmaceutically acceptable salt thereof. In an embodiment, the xanthone is 1,3,7-trihydroxy-2,4-diisoprenylxanthone or a pharmaceutically acceptable salt thereof. In an embodiment, the xanthone is cratoxyarborenone B or a pharmaceutically acceptable salt thereof. In an embodiment, the xanthone is 1,3,5-trihydroxy-2-prenyl-9H-xanthene-9-one or a pharmaceutically acceptable salt thereof. In an embodiment, the xanthone is 1,3,7-trihydroxy-2-prenylxanthone or a pharmaceutically acceptable salt thereof. In an embodiment, the xanthone is doitunggarcinone C or a pharmaceutically acceptable salt thereof. In an embodiment, the xanthone is cudratricusxanthone K or a pharmaceutically acceptable salt thereof. In an embodiment, the xanthone is 1-hydroxy-3,5-dimethoxy-2-prenylxanthone or a pharmaceutically acceptable salt thereof. In an embodiment, the xanthone is 1,8-dihydroxy-3,5-dimethoxy-2-prenylxanthone or a pharmaceutically acceptable salt thereof.

In an embodiment, the xanthone is alpha-mangostin or a pharmaceutically acceptable salt thereof, provided substantially free of another xanthone such as beta-mangostin, gamma-mangostin, or 8-deoxygartanin. In an embodiment, the xanthone is beta-mangostin or a pharmaceutically acceptable salt thereof, provided substantially free of another xanthone such as alpha-mangostin, gamma-mangostin, or 8-deoxygartanin. In an embodiment, the xanthone is gamma-mangostin or a pharmaceutically acceptable salt thereof, provided substantially free of another xanthone such as alpha-mangostin, beta-mangostin, or 8-deoxygartanin. In an embodiment, the xanthone is 8-deoxygartanin or a pharmaceutically acceptable salt thereof, provided substantially free of another xanthone such as alpha-mangostin, beta-mangostin, or gamma-mangostin.

In an embodiment, the xanthone is alpha-mangostin or a pharmaceutically acceptable salt thereof, provided substantially free of another xanthone shown in Table 2. In an embodiment, the xanthone is beta-mangostin or a pharmaceutically acceptable salt thereof, provided substantially free of another xanthone shown in Table 2. In an embodiment, the xanthone is gamma-mangostin or a pharmaceutically acceptable salt thereof, provided substantially free of another xanthone shown in Table 2. In an embodiment, the xanthone is 8-deoxygartanin or a pharmaceutically acceptable salt thereof, provided substantially free of another xanthone s shown in Table 2.

In an embodiment, the xanthone or a pharmaceutically acceptable salt thereof is provided as a substantially pure compound, for example, at least 85%, at least 90%, at least 95%, at least 98%, at least 99%, or at least 99.9% pure, measured, e.g, by HPLC analysis. In an embodiment, the xanthone is gamma-mangostin or a pharmaceutically acceptable salt thereof, and is provided as a substantially pure compound, for example, at least 85%, at least 90%, at least 95%, at least 98%, at least 99%, or at least 99.9% pure, measured, e.g, by HPLC analysis.

In an embodiment, the xanthone is provided with less than 10%, 7.5%, 5%, 2.5%, 1%, 0.5%, or 0.1% of another xanthone present. In an embodiment, the xanthone is gamma-mangostin and is provided with less than 10%, 7.5%, 5%, 2.5%, 1%, 0.5%, or 0.1% of another xanthone present. In an embodiment, the xanthone is gamma-mangostin and is provided with less than 10%, 7.5%, 5%, 2.5%, 1%, 0.5%, or 0.1% of alpha-mangostin or beta-mangostin present. In an embodiment, the xanthone is provided with less than 10%, 7.5%, 5%, 2.5%, 1%. 0.5%, or 0.1% of another plant material, such as a plant metabolite or plant lipid. In an embodiment, the xanthone is gamma-mangostin and is provided with less than 10%, 7.5%, 5%, 2.5%, 1%, 0.5%, or 0.1% of another plant material, such as a plant metabolite or plant lipid.

A composition useful for the treatment of inflammation or an inflammatory disease or disorder may contain a xanthone (e.g., gamma-mangostin) or a plurality of xanthones. For example, a composition comprising a xanthone may contain only gamma-mangostin, e.g., synthetically prepared or extracted from a natural source, in the absence of another xanthone. In contrast, a composition comprising a xanthone may also contain a combination of gamma-mangostin and a closely related analog or variant thereof, e.g., a xanthone shown in Table 1, e.g., alpha-mangostin or beta-mangostin.

The flavonoids and xanthones provided herein may contain one or more asymmetric centers and thus occur as racemates and racemic mixtures, single enantiomers, individual diastereomers and diastereomeric mixtures. Further, the flavonoids and xanthones may exist as one of many tautomeric forms. All such isomeric and tautomeric forms of these compounds are expressly included within the scope. Unless otherwise indicated when a compound is named or depicted by a structure without specifying the stereochemistry and has one or more chiral centers or tautomeric structures, it is understood to represent all possible stereoisomers or tautomers of the compound. The compounds provided herewith may also contain linkages (e.g., carbon-carbon bonds, phosphorus-oxygen bonds, or phosphorus-sulfur bonds) or substituents that can restrict bond rotation, e.g., restriction resulting from the presence of a ring or double bond.

### Inflammatory Diseases and Disorders

Described herein are methods for treating inflammation or an inflammatory disease or disorder in a subject, tissue, or cell, e.g., by administration of a xanthone and a flavonoid. In embodiments, the cell is an isolated cell (e.g., a cell in a cell culture or a cell isolated from a tissue or intact organism). In embodiments, the cell is located in a tissue or organ.

An inflammatory disease or disorder relates to any disease or disorder that affects the immune system and may result in a modulation of the inflammatory pathway. Exemplary inflammatory diseases and disorders include dermatological conditions (e.g., psoriasis, dermal hypersensitivity, eczema, burns, atopic dermatitis, or abnormal proliferation of hair follicle cells), fibrosis (e.g., kidney or lung fibrosis), allergic rhinitis, respiratory distress syndrome, asthma, bronchitis, tendinitis, bursitis, fever, migraine headaches, gastrointestinal conditions (e.g., inflammatory bowel disease, Crohns disease, gastritis, irritable bowel syndrome, colitis and colorectal cancer), vascular diseases (e.g., atherosclerosis), periarteritis nodosa, thyroiditis, aplastic anemia, Hodgkins Disease, rheumatic fever, osteoarthritis, autoimmune diseases (e.g., type I diabetes, myasthenia gravis, rheumatoid arthritis, systemic lupus erythematosus, and multiple sclerosis), sarcoidosis, nephrotic syndrome, renal failure, Behcets Syndrome, polymyositis, gingivitis, hypersensitivity (e.g., delayed type hypersentivity or immediate hypersensitivity), graft and transplant rejections, graft versus host disease (GVHD), conjunctivitis, swelling occurring after injury, myocardial ischemia, and endotoxin shock syndrome.

Psoriasis is a chronic skin disorder that afflicts about 2 percent of the population. The disease is associated with the rapid turnover of skin cells (hyperproliferation) accompanied by a loss of differentiation so that silvery white scales form on the surface of the skin. Additionally, the capillaries become tortuous and dilated and an inflammatory reaction occurs, so that the skin reddens. The elevated silvery white scales on a contrasting red background produce the unsightly lesions characteristic of psoriasis. Psoriasis most commonly appears on the scalp, knees, elbows, hands and feet, but can affect any part of the skin. The cause of the disease is unknown, though it is believed to have a genetic component, and it has been suggested to be a T-cell mediated autoimmune skin disorder. In an embodiment, the inflammatory disease or disorder comprises psoriasis.

Dermal hypersensitivity is a nonspecific histological reaction pattern that may be seen in multiple clinical conditions. Symptoms may include pruritis, lesions, and in idiopathic cases, urticarial or eczematous papules or plaques. In an embodiment, the inflammatory disease or disorder comprises dermal hypersensitivity.

Eczema is a general term for many types of skin inflammation, also known as dermatitis. The most common form of eczema is atopic eczema or dermatitis. Eczema occurs in people of all races and can affect people of any age, although the condition is most common in infants, and about 85% of people have an onset prior to five years of age. Typically, eczema will permanently resolve by age three in only about one-half of affected infants. In others, the condition tends to recur throughout life. People with eczema often have a family history of the condition or a family history of other allergic conditions, such as asthma and/or hay fever. While eczema is not known to be contagious, it is believed to be at least partially inherited. In an embodiment, the inflammatory disease or disorder comprises eczema.

Approximately 140 million people suffer burns per year in the United States alone. Of these, 54,000 to 180,000 people are estimated to be hospitalized. A burn is an injury to the skin or other tissue caused by heat, cold, electricity, chemicals, friction, or radiation. Most burns are caused by heat generated by hot liquids (burns), solids, or fire. The skin is composed of three major tissue layers: epidermis, dermis, and subcutaneous tissue. Burns that affect only the outermost skin are called superficial or first-order burns. They appear red, free of blisters, and pain usually lasts for about three days. When the damage extends to some underlying skin layer, it is called a local thickness or secondary burn. Blisters often occur and are often painful. Healing may take up to eight weeks and scarring may occur. In full thickness or third degree burns, the damage extends to all layers of the skin. Usually without pain and the burned area is stiff. It does not heal itself normally and skin grafting is required. Fourth degree burns also involve damage to deeper tissues such as muscles, tendons or bones. Such burns are usually black and often result in loss of the burn site. In an embodiment, the inflammatory disease or disorder comprises burns.

Atopic dermatitis is the most common form of eczema. It is a condition that causes dry, itchy and inflamed skin. It is common in young children but can occur at any age. Atopic dermatitis is a chronic condition that can cause flare ups throughout a patient's life and is not contagious. People with atopic dermatitis are at risk of developing food allergies, hay fever, and asthma. Symptoms of atopic dermatitis may include dry, cracked skin, itchiness (pruritis), small, raised bumps, thickened skin, darkening of the skin around the eyes, and raw, sensitive skin. In an embodiment, the inflammatory disease or disorder comprises atopic dermatitis.

Fibrosis is a process characterized by an excessive accumulation of the extracellular matrix as a response to different types of tissue injuries, which leads to organ dysfunction. The process can be initiated by multiple and different stimuli and pathogenic factors which trigger the cascade of reparation converging in molecular signals responsible of initiating and driving fibrosis. Though fibrosis can play a defensive role, in several circumstances at a certain stage, it can progressively become an uncontrolled irreversible and self-maintained process, named pathological fibrosis. Such fibrosis can occur in any organ in the body, e.g., in the kidney(s) (kidney fibrosis), or in the lung(s) (lung fibrosis). In an embodiment, the inflammatory disease or disorder comprises fibrosis, e.g., kidney fibrosis and lung fibrosis.

Allergic rhinitis, also called hay fever, is an allergic reaction that causes sneezing, congestion, itchy nose and sore throat. Pollen, pet dander, mold and insects can lead to hay fever symptoms. Symptoms of hay fever include sneezing, nasal congestion and irritation of the nose, throat, mouth and eyes. In an embodiment, the inflammatory disease or disorder comprises allergic rhinitis.

Respiratory distress syndrome is a disease involving increased pulmonary capillary permeability. The consequent accumulation of protein-rich fluid inside the alveoli is the result of the damage to the capillary endothelium and alveolar epithelium; this causes the release of cytokines, producing diffuse alveolar damage. Common risk factors for ARDS include: pneumonia, sepsis, gastric content aspiration, trauma, pancreatitis, inhalation injury, burns, non-cardiogenic shock, drug overdose, transfusion related acute lung injury (TRALI), and drowning. The characteristic pathological features of ARDS have classically been described by three overlapping phases: an exudative or inflammatory phase, a proliferative phase, and a fibrotic phase. In an embodiment, the inflammatory disease or disorder comprises respiratory distress syndrome.

Asthma is a chronic inflammatory disease of the airways characterized by airway hyper responsiveness, acute and chronic bronchoconstriction, airway edema, and mucus plugging. The inflammation component of asthma is thought to involve many cell types, including mast cells, eosinophils, T lymphocytes, neutrophils, and epithelial cells, and their biological products. Patients with asthma most often present with symptoms of wheezing, shortness of breath, cough, and chest tightness. In an embodiment, the inflammatory disease or disorder comprises asthma.

Bronchitis is an inflammation of the mucous membrane of the trachea and bronchi caused by bacterial and viral infections or irritations caused by physical and chemical factors. As a rule, this disease is mainly characterized by cough, sputum, discomfort or pain behind the sternum, shortness of breath and the usual cold symptoms that accompany them. In accordance with the duration of bronchitis can be divided into two forms: acute tracheobronchitis and chronic bronchitis. Chronic bronchitis is bronchitis which lasts more than two months and worsens for two consecutive years or lasts three consecutive months for one year, causing inflammation of the mucous membrane and peripheral tissues. Most patients are adults, and the incidence of bronchitis increases in the winter and spring. In an embodiment, the inflammatory disease or disorder comprises bronchitis.

Tendinitis is a tendon inflammation referring to the inflammatory changes of the tendon tissue. It often results in degenerative changes of the tissue affected, possibly including calcium deposits at a later time. In general, tendinitis can affect any tendon of the body. Since tendinitis is mainly caused by mechanical stress (sports), tendinitis affects in particular certain body regions, such as e.g., in the region of the shoulder, the tibia, or the foot. Tendinitis can also occur in the context of inflammatory rheumatoid diseases (in particular Reiters syndrome, spondylitis ankylosans, and arthritis psoriatica). In an embodiment, the inflammatory disease or disorder comprises tendinitis.

Bursitis is an inflammation of a bursa or protecting sac that is located in or near the patients joints. Bursitis can be acute resulting in a sudden, sharp pain following an injury, or it can be chronic as a result of a recurrent inflammation in the same area. Most commonly, bursitis is caused by trauma, infection and crystal deposits. Bursitis typically results from overuse or injury of a joint as a consequence of intensive working or playing, poor conditioning before exercising or sporting, systematic incorrect posture at work or rest, or an abnormal positioned joint as a result of for instance arthritis or bone length differences that stress soft tissue structures in the human or animal body. In an embodiment, the inflammatory disease or disorder comprises bursitis.

Fever is a commonly used medical indicator characterized by elevation of body temperature above a normal range. In an embodiment, the inflammatory disease or disorder comprises fever.

Migraine comprises a complex, common neurological condition that is characterized by severe, episodic attacks of headache and associated features, which may include nausea, vomiting, sensitivity to light, sound or movement. In some patients, the headache is preceded or accompanied by sensory warning signs or symptoms (i.e., auras). The headache pain may be severe and may also be unilateral in certain patients. Migraine attacks are disruptive to daily life and cost billions of dollars each year in missed work days and impaired performance. Migraine is a highly prevalent disease worldwide with approximately 15% of the European population and 12% of the United States population suffering from migraine attacks. Additionally, migraines have been found to be associated with a number of psychiatric and medical comorbidities such as depression and vascular disorders. In an embodiment, the inflammatory disease or disorder comprises migraine headaches.

Inflammatory bowel disease (TBD) is a chronic inflammatory autoimmune condition of the gastrointestinal (GI) tract, which presents clinically as either ulcerative colitis (UC) or Crohns disease (CD). CD is a chronic transmural inflammatory disease with the potential to affect any part of the entire GI tract, and UC is a mucosal inflammation of the colon. Both conditions are characterized clinically by frequent bowel motions, malnutrition, and dehydration, with disruption in the activities of daily living. CD is frequently complicated by the development of malabsorption, strictures, and fistulae and may require repeated surgery. UC, less frequently, may be complicated by severe bloody diarrhea and toxic megacolon, also requiring surgery. Both IBD conditions are associated with an increased risk for malignancy of the GI tract. The etiology of IBD is complex, and many aspects of the pathogenesis remain unclear. In an embodiment, the inflammatory disease or disorder comprises inflammatory bowel disease (e.g., Crohn's disease or ulcerative colitis).

Gastritis is a general term for a group of conditions comprising inflammation of the stomach lining. The inflammation of gastritis is most often the result of infection with the same bacterium that causes most stomach ulcers or the regular use of certain pain relievers. Drinking too much alcohol also can contribute to gastritis. Gastritis may occur suddenly (acute gastritis) or appear slowly over time (chronic gastritis). In some cases, gastritis can lead to ulcers and an increased risk of stomach cancer. In an embodiment, the inflammatory disease or disorder comprises gastritis.

Irritable bowel syndrome (IBS) is the most common of all gastrointestinal disorders, affecting 11-14% of adults and accounting for more than 50% of all patients with digestive complaints. It is thought that only a minority of people with IBS actually seek medical treatment. Patients with IBS present with disparate symptoms, for example, abdominal pain predominantly related to defecation, alternating diarrhea and constipation, abdominal distention, gas, and excessive mucus in the stool. A number of possible causes for IBS have been proposed, but none has been fully accepted. These hypotheses included a fiber-poor Western diet, intestinal motility malfunction, abnormal pain perception, abnormal psychology or behavior, or psychophysiological response to stress. In an embodiment, the inflammatory disease or disorder comprises TBS.

Colorectal cancer (CRC), also known as bowel cancer, colon cancer, or rectal cancer, is the development of cancer from the colon or rectum (parts of the large intestine). Signs and symptoms may include blood in the stool, a change in bowel movements, weight loss, and fatigue. Most colorectal cancers are due to old age and lifestyle factors, with only a small number of cases due to underlying genetic disorders. Risk factors include diet, obesity, smoking, and lack of physical activity. Another risk factor is inflammatory bowel disease, which includes Crohns disease and ulcerative colitis. Some of the inherited genetic disorders that can cause colorectal cancer include familial adenomatous polyposis and hereditary non-polyposis colon cancer; however, these represent less than 5% of cases. It typically starts as a benign tumor, often in the form of a polyp, which over time becomes cancerous. In an embodiment, the inflammatory disease or disorder comprises colorectal cancer.

Atherosclerosis is a chronic disease that causes thickening of the innermost layer (intima) of the aorta or middle artery. This reduces blood flow and can cause ischemia and tissue destruction in organs supplied by affected blood vessels. Atherosclerosis is a major cause of heart disease including myocardial infarction, stroke and surrounding arterial disease. The disease begins with the accumulation of lipoproteins, mainly low density lipoprotein (LDL), in the extracellular matrix of blood vessels. These LDL particles aggregate and promote oxidative modification. Oxidized LDL is toxic and causes vascular damage. Atherosclerosis represents a response to this wound in the form of inflammation and fibrosis. In an embodiment, the inflammatory disease or disorder comprises atherosclerosis.

Polyarteritis nodosa (PAN) is a systemic necrotizing inflammation of blood vessels (vasculitis) affecting medium-sized muscular arteries, typically involving the arteries of the kidneys and other internal organs but generally sparing the lungs circulation. Small aneurysms are strung like the beads of a rosary, therefore making this “rosary sign” an important diagnostic feature of the vasculitis. PAN is sometimes associated with infection by the hepatitis B or hepatitis C virus. The condition may be present in infants. In an embodiment, the inflammatory disease or disorder comprises periarteritis nodosa.

Thyroiditis is the inflammation of the thyroid gland. There are many different signs and symptoms for thyroiditis, none of which are exclusively limited to this disease. Many of the signs imitate symptoms of other diseases, so thyroiditis can sometimes be difficult to diagnose. Common hypothyroid symptoms manifest when thyroid cell damage is slow and chronic, and may include fatigue, weight gain, feeling “fuzzy headed”, depression, dry skin, and constipation. Other, rarer symptoms include swelling of the legs, vague aches and pains, decreased concentration and so on. If the thyroid cell damage is acute, the thyroid hormone within the gland leaks out into the bloodstream causing symptoms of thyrotoxicosis, which is similar to those of hyperthyroidism. These symptoms include weight loss, irritability, anxiety, insomnia, fast heart rate, and fatigue. Elevated levels of thyroid hormone in the bloodstream cause both conditions, but thyrotoxicosis is the term used with thyroiditis since the thyroid gland is not overactive, as in the case of hyperthyroidism. In an embodiment, the inflammatory disease or disorder comprises thyroiditis.

Aplastic anemia is a severe hematologic condition in which the body fails to make blood cells in sufficient numbers. Aplastic anemia is associated with cancer and various cancer syndromes. Blood cells are produced in the bone marrow by stem cells that reside there. Aplastic anemia causes a deficiency of all blood cell types: red blood cells, white blood cells, and platelets. It occurs most frequently in people in their teens and twenties but is also common among the elderly. It can be caused by heredity, immune disease, or exposure to chemicals, drugs, or radiation. However, in about half of cases, the cause is unknown. In an embodiment, the inflammatory disease or disorder comprises aplastic anemia.

Hodgkin's disease, also known as Hodgkin's lymphoma (HL) is a type of lymphoma in which cancer originates from a specific type of white blood cell called lymphocytes, where multinucleated Reed-Sternberg cells (RS cells) are present in the patients lymph nodes. Symptoms may include fever, night sweats, and weight loss. Often, nonpainful enlarged lymph nodes occur in the neck, under the arm, or in the groin. About half of cases of Hodgkin lymphoma are due to Epstein-Barr virus (EBV) and these are generally the classic form. Other risk factors include a family history of the condition and having HIV/AIDS. In an embodiment, the inflammatory disease or disorder comprises Hodgkin's disease.

Rheumatic fever (RF) is an inflammatory disease that can involve the heart, joints, skin, and brain. The disease typically develops two to four weeks after a streptococcal throat infection. Signs and symptoms include fever, multiple painful joints, involuntary muscle movements, and occasionally a characteristic non-itchy rash known as erythema marginatum. The heart is involved in about half of the cases. Damage to the heart valves, known as rheumatic heart disease (RHD), usually occurs after repeated attacks but can sometimes occur after one. The damaged valves may result in heart failure, atrial fibrillation and infection of the valves. In an embodiment, the inflammatory disease or disorder comprises rheumatic fever.

Osteoarthritis (OA) is a type of degenerative joint disease that results from breakdown of joint cartilage and underlying bone which affects 1 in 7 adults in the United States. It is believed to be the fourth leading cause of disability in the world. The most common symptoms are joint pain and stiffness. Other symptoms may include joint swelling, decreased range of motion, and, when the back is affected, weakness or numbness of the arms and legs. The most commonly involved joints are the two near the ends of the fingers and the joint at the base of the thumbs, the knee and hip joints, and the joints of the neck and lower back. Unlike some other types of arthritis, only the joints, not internal organs, are affected. In an embodiment, the inflammatory disease or disorder comprises osteoarthritis.

Type 1 diabetes (T1D), formerly known as juvenile diabetes, is an autoimmune disease that originates when cells that make insulin (beta cells) are destroyed by the immune system, resulting in high blood sugar levels. The common symptoms of this elevated blood sugar are frequent urination, increased thirst, increased hunger, weight loss, and other serious complications. Additional symptoms may include blurry vision, tiredness, and slow wound healing. Symptoms typically develop over a short period of time, often a matter of weeks. The underlying mechanism involves an autoimmune destruction of the insulin-producing beta cells in the pancreas. In an embodiment, the inflammatory disease or disorder comprises Type I diabetes.

Myasthenia gravis (MG) is a long-term autoimmune neuromuscular junction disease that leads to varying degrees of skeletal muscle weakness. The most commonly affected muscles are those of the eyes, face, and swallowing. It can result in double vision, drooping eyelids, trouble talking, and trouble walking. Onset can be sudden. Those affected often have a large thymus or develop a thymoma. In an embodiment, the inflammatory disease or disorder comprises myasthenia gravis.

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body, including skin, eyes, lungs, heart, nerves and blood. This may result in a low red blood cell count, inflammation around the lungs, and inflammation around the heart. Fever and low energy may also be present. In an embodiment, the inflammatory disease or disorder comprises rheumatoid arthritis.

Lupus, technically known as systemic lupus erythematosus (SLE), is an autoimmune disease in which the body s immune system mistakenly attacks healthy tissue in many parts of the body. Symptoms vary among people and may be mild to severe. Common symptoms include painful and swollen joints, fever, chest pain, hair loss, mouth ulcers, swollen lymph nodes, feeling tired, and a red rash which is most commonly on the face. Often there are periods of illness, called flares, and periods of remission during which there are few symptoms. The cause of SLE is not clear. It is thought to involve a combination of genetics and environmental factors. In an embodiment, the inflammatory disease or disorder comprises systemic lupus erythematosus.

Multiple sclerosis (MS) is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, visual loss, muscle weakness, and trouble with sensation or coordination. MS takes several forms, with new symptoms either occurring in isolated attacks (relapsing forms) or building up over time (progressive forms). While the cause is unclear, the underlying mechanism is thought to be either destruction by the immune system or failure of the myelin-producing cells. In an embodiment, the inflammatory disease or disorder comprises multiple sclerosis.

Sarcoidosis is a disease involving abnormal collections of inflammatory cells that form lumps known as granulomata. The disease usually begins in the lungs, skin, or lymph nodes. Less commonly affected are the eyes, liver, heart, and brain, though any organ can be affected. The signs and symptoms depend on the organ involved. Often, no, or only mild, symptoms are seen. When it affects the lungs, wheezing, coughing, shortness of breath, or chest pain may occur. In an embodiment, the inflammatory disease or disorder comprises sarcoidosis.

Nephrotic syndrome is a collection of symptoms due to kidney damage. This includes protein in the urine, low blood albumin levels, high blood lipids, and significant swelling. Other symptoms may include weight gain, feeling tired, and foamy urine. Complications may include blood clots, infections, and high blood pressure. Causes include a number of kidney diseases such as focal segmental glomerulosclerosis, membranous nephropathy, and minimal change disease. It may also occur as a complication of diabetes or lupus. Tn an embodiment, the inflammatory disease or disorder comprises nephrotic syndrome.

Kidney failure, also known as end-stage kidney disease, is a medical condition in which the kidneys can no longer adequately filter waste products from the blood, functioning at less than 15% of normal levels. Kidney failure is classified as either acute kidney failure, which develops rapidly and may resolve; and chronic kidney failure, which develops slowly and can often be irreversible. Symptoms may include leg swelling, feeling tired, vomiting, loss of appetite, and confusion. In an embodiment, the inflammatory disease or disorder comprises renal failure.

Behçets disease (BD) is a type of inflammatory disorder which affects multiple parts of the body. The most common symptoms include painful sores on the mucous membranes of the mouth and other parts of the body, inflammation of parts of the eye, and arthritis. The sores can last from a few days, up to a week or more. Less commonly there may be inflammation of the brain or spinal cord, blood clots, aneurysms, or blindness. Often, the symptoms come and go. In an embodiment, the inflammatory disease or disorder comprises Behcet's disease.

Polymyositis is a type of chronic inflammation of the muscles (inflammatory myopathy) related to dermatomyositis and inclusion body myositis. The inflammation of polymyositis is mainly found in the endomysial layer of skeletal muscle, whereas dermatomyositis is characterized primarily by inflammation of the perimysial layer of skeletal muscles. In an embodiment, the inflammatory disease or disorder comprises polymyositis.

Gingivitis is a non-destructive disease that causes inflammation of the gums. The most common form of gingivitis, and the most common form of periodontal disease overall, is in response to bacterial biofilms (also called plaque) that is attached to tooth surfaces, termed plaque-induced gingivitis. Most forms of gingivitis are plaque-induced. In an embodiment, the inflammatory disease or disorder comprises gingivitis.

Graft-versus-host disease (GVHD) is a syndrome, characterized by inflammation in different organs. GVHD is commonly associated with bone marrow transplants and stem cell transplants. White blood cells of the donors immune system which remain within the donated tissue (the graft) recognize the recipient (the host) as foreign (non-self). The white blood cells present within the transplanted tissue then attack the recipients bodys cells, which leads to GVHD. This should not be confused with a transplant rejection, which occurs when the immune system of the transplant recipient rejects the transplanted tissue; GvHD occurs when the donors immune systems white blood cells reject the recipient. In an embodiment, the inflammatory disease or disorder comprises GVHD.

Conjunctivitis, also known as pink eye, is inflammation of the outermost layer of the white part of the eye and the inner surface of the eyelid. It makes the eye appear pink or reddish. Pain, burning, scratchiness, or itchiness may occur. The affected eye may have increased tears or be “stuck shut” in the morning. Swelling of the white part of the eye may also occur. Itching is more common in cases due to allergies. Conjunctivitis can affect one or both eyes. In an embodiment, the inflammatory disease or disorder comprises conjunctivitis.

Myocardial ischemia involves the reduction of blood flow to the heart muscle due to build-up of atherosclerotic plaque in the arteries of the heart. It is the most common of the cardiovascular diseases. A common symptom is chest pain or discomfort which may travel into the shoulder, arm, back, neck, or jaw. Occasionally it may feel like heartburn. Usually symptoms occur with exercise or emotional stress, last less than a few minutes, and improve with rest. Shortness of breath may also occur and sometimes no symptoms are present. In many cases, the first sign is a heart attack. In an embodiment, the inflammatory disease or disorder comprises myocardial ischemia.

Endotoxin shock syndrome, also known as toxic shock syndrome (TSS), is an uncommon but life-threatening disease which is caused by the poisonous endotoxin (TSS-1) produced by Staphylococcus aureus bacteria. Infection is caused due to the entrance of bacteria in the body through skin opening such as cuts or wounds or due to poorly conducted skin surgery, skin-burn, and skin infection. Prevalence of S. aureus colonization and antibodies by age, geographical area there was no significant difference found in the rate of toxigenicity and colonization while the population study prevalence was found to be 26%. Symptoms of TSS include skin rashes, headache, fever, vomiting, diarrhea, including neurological disturbance and the central nervous system disturbance along with organ failure. In an embodiment, the inflammatory disease or disorder comprises endotoxin shock syndrome.

In an embodiment, the inflammatory disease or disorder is a dermatological condition. For example, the dermatological disease or disorder may be atopic dermatitis, psoriasis, eczema, or dermal hypersensitivity. In an embodiment, the dermatological condition is atopic dermatitis. In an embodiment, the dermatological condition is psoriasis. In an embodiment, the dermatological condition is eczema. In an embodiment, the dermatological condition is dermal hypersensitivity.

### Combination Therapies

Described herein are combination therapies comprising a flavonoid and a xanthone useful for the treatment of inflammation or an inflammatory disease or disorder. The combination therapy may be administered as a single formulation or as separate formulations. In an embodiment, the flavonoid and xanthone are administered as a single pharmaceutical composition. In an embodiment, the flavonoid and xanthone are administered as separate pharmaceutical compositions. In the case of separate formulations, the flavonoid and xanthone may be administered concomitantly or sequentially. In an embodiment, the flavonoid and xanthone are administered concomitantly. In an embodiment, the flavonoid and xanthone are administered sequentially. For example, the flavonoid may be administered prior to the xanthone or subsequent to the xanthone.

In some embodiments, the administration of the flavonoid and xanthone has a synergistic or additive effect. For example, the administration of the flavonoid and xanthone may have an additive effect, in which the therapeutic effect of the flavonoid and xanthone is the total sum of the effects of each of the components individually. In contrast, the administration of the flavonoid and xanthone may have a synergistic effect, in which the therapeutic effect of the flavonoid and xanthone is greater than the sum of the individual components. The synergistic effect of the combination of the flavonoid and xanthone may be 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 95%, or more than the total sum of the effects of the flavonoid and xanthone administered individually. In an embodiment, the synergistic effect of the combination of flavonoid and xanthone is greater than between 5% and 75% of the total sum of the effects of the flavonoid and xanthone administered individually.

In an embodiment, the synergistic effect of the combination of the flavonoid and xanthone is greater than 10% of the total sum of the effects of the flavonoid and xanthone administered individually. In an embodiment, the synergistic effect of the combination of the flavonoid and xanthone is greater than 25% of the total sum of the effects of the flavonoid and xanthone administered individually. In an embodiment, the synergistic effect of the combination of the flavonoid and xanthone is greater than 50% of the total sum of the effects of the flavonoid and xanthone administered individually. In an embodiment, the synergistic effect of the combination of the flavonoid and xanthone is greater than 75% of the total sum of the effects of flavonoid and xanthone administered individually.

The synergistic effect of the combination of the flavonoid and xanthone may be 1.5-fold, 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 75-fold, 100-fold, 200-fold, 500-fold greater or more than the total sum of the effects of the flavonoid and xanthone administered individually. In an embodiment, the synergistic effect of the combination of the flavonoid and xanthone is greater than between 2-fold and 100-fold of the total sum of the effects of the flavonoid and xanthone administered individually. In an embodiment, the synergistic effect of the combination of the flavonoid and xanthone is greater than between 5-fold and 50-fold of the total sum of the effects of the flavonoid and xanthone administered individually.

In one aspect, the amount of the flavonoid and the amount of the xanthone are selected such that the molar concentration of the flavonoid is greater than the molar concentration of the xanthone. For example, the molar concentration of the flavonoid is greater than 1.5-fold, 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 75-fold, 100-fold, 200-fold, or 500-fold greater than the molar concentration of the xanthone. In an embodiment, the molar concentration of the flavonoid is greater than 1.5-fold, 2-fold, 3-fold, 4-fold, 5-fold, 6-fold, 7-fold, 8-fold, 9-fold, or 10-fold greater than the molar concentration of the xanthone. In an embodiment, the molar concentration of the flavonoid is between 2-fold and 20-fold greater than the molar concentration of the xanthone. In an embodiment, the molar concentration of the flavonoid is between 5-fold and 10-fold greater than the molar concentration of the xanthone. In an embodiment, the molar concentration of the flavonoid is about 5-fold greater than the molar concentration of the xanthone. In an embodiment, the molar concentration of the flavonoid is about 10-fold greater than the molar concentration of the xanthone. In an embodiment, the molar concentration of the flavonoid is about 15-fold greater than the molar concentration of the xanthone.

In another aspect, the combination of the flavonoid with a xanthone results in reducing inflammation (e.g., the level of an inflammatory cytokine, e.g., IL-6, IL-1b, and TNF-a) in a subject or cell. For example, administration of a combination of the flavonoid with a xanthone may result reducing inflammation (e.g., the level of an inflammatory cytokine, e.g., IL-6, IL-1b, and TNF-a) in a subject or cell, compared with the inflammation (e.g., the level of an inflammatory cytokine, e.g., IL-6, IL-1b, and TNF-a) that results when the xanthone is administered to the subject or cell individually. In an embodiment, the inflammation (e.g., the level of an inflammatory cytokine, e.g., TL-6, IL-1b, and TNF-a) in a subject or cell is reduced upon administration of the xanthone in combination with a flavonoid, e.g., by about 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 95%, or more compared with the inflammation (e.g., the level of an inflammatory cytokine, e.g., IL-6, IL-1b, and TNF-a) that results when a xanthone is administered individually.

In another aspect, the combination of the flavonoid with a xanthone results in reducing inflammation (e.g., the level of an inflammatory cytokine, e.g., IL-6, IL-1b, and TNF-a) in a subject or cell. For example, administration of a combination of the flavonoid with a xanthone may result in reducing the inflammation (e.g., the level of an inflammatory cytokine, e.g., IL-6, IL-1b, and TNF-a) in a subject or cell, compared with the inflammation (e.g., the level of an inflammatory cytokine, e.g., IL-6, IL-1b, and TNF-a) that results when the flavonoid is administered to the subject or cell individually. In an embodiment, the inflammation (e.g., the level of an inflammatory cytokine, e.g., IL-6, IL-1b, and TNF-a) in a subject or cell is reduced upon administration of a xanthone in combination with a flavonoid, e.g., by about 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 95%, or more compared with the inflammation (e.g., the level of an inflammatory cytokine) that results when the flavonoid is administered individually.

In another aspect, the combination of the flavonoid with a xanthone results in increasing the level of an anti-inflammatory cytokine (e.g., IL-4 and IL-10) in a subject or cell. For example, administration of a combination of the flavonoid with a xanthone may result increasing the level of an anti-inflammatory cytokine (e.g., IL-4 and IL-10) in a subject or cell, compared with the level of an anti-inflammatory cytokine (e.g., IL-4 and IL-10) that results when the xanthone is administered to the subject or cell individually. In an embodiment, the level of an anti-inflammatory cytokine (e.g., IL-4 and IL-10) in a subject or cell is increased upon administration of the xanthone in combination with a flavonoid, e.g., by about 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 95%, or more compared with the level of an anti-inflammatory cytokine (e.g., IL-4 and IL-10) that results when a xanthone is administered individually.

In another aspect, the combination of the flavonoid with a xanthone results in increasing the level of an anti-inflammatory cytokine (e.g., IL-4 and IL-10) in a subject or cell. For example, administration of a combination of the flavonoid with a xanthone may result in increasing the level of an anti-inflammatory cytokine (e.g., IL-4 and IL-10) in a subject or cell, compared with level of an anti-inflammatory cytokine that results when the flavonoid is administered to the subject or cell individually. In an embodiment, the level of an anti-inflammatory cytokine (e.g., IL-4 and IL-10) in a subject or cell is increased upon administration of a xanthone in combination with a flavonoid, e.g., by about 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 95%, or more compared with the level of an inflammatory cytokine that results when the flavonoid is administered individually.

In an embodiment, the flavonoid is administered at a dosage to provide a concentration of between 0.1 μM and 500 μM in a subject or cell. In an embodiment, the flavonoid is administered at a dosage to provide a concentration of between 0.5 μM and 100 μM in a subject or cell. In an embodiment, the flavonoid is administered at a dosage to provide a concentration of between 1 μM and 100 μM in a subject or cell. In an embodiment, the flavonoid is administered at a dosage to provide a concentration of between 1 μM and 50 μM in a subject or cell. In an embodiment, the flavonoid is administered at a dosage to provide a concentration of between 1 μM and 25 μM in a subject or cell. In an embodiment, the xanthone is administered at a dosage to provide a concentration of between 0.01 μM and 50 μM in a subject or cell. In an embodiment, the xanthone is administered at a dosage to provide a concentration of between 0.1 μM and 25 μM in a subject or cell. In an embodiment, the xanthone is administered at a dosage to provide a concentration of between 0.1 μM and 10 μM in a subject or cell. In an embodiment, the xanthone is administered at a dosage to provide a concentration of between 0.1 μM and 5 μM in a subject or cell. In an embodiment, the xanthone is administered at a dosage to provide a concentration of between 0.1 μM and 2.5 μM in a subject or cell.

In an embodiment, the flavonoid is administered at a dosage to provide a concentration of between 0.1 μM and 500 μM and the xanthone is administered at a dosage to provide a concentration of between 0.01 μM and 50 μM in a subject or cell. In an embodiment, the flavonoid is administered at a dosage to provide a concentration of to provide a concentration of between 1 μM and 100 μM and the xanthone is administered at a dosage to provide a concentration of between 0.1 μM and 25 μM in a subject or cell. In an embodiment, the flavonoid is administered at a dosage to provide a concentration of to provide a concentration of between 1 μM and 25 μM and the xanthone is administered at a dosage to provide a concentration of between 0.1 μM and 2.5 μM in a subject or cell.

### Pharmaceutical Compositions

The present disclosure features methods for treating a subject having inflammation or an inflammatory disease or disorder, the methods comprising administering a flavonoid, a xanthone, or a combination thereof.

While it is possible for a flavonoid (e.g., a compound of Table 1) or a xanthone (e.g, a compound of Table 2) to be administered alone, it is preferable to administer said compound as a pharmaceutical composition or formulation, where the compounds are combined with one or more pharmaceutically acceptable diluents, excipients or carriers. The compounds according to the disclosure may be formulated for administration in any convenient way for use in human or veterinary medicine. In certain embodiments, the compounds included in the pharmaceutical preparation may be active itself, or may be a prodrug, e.g., capable of being converted to an active compound in a physiological setting. Regardless of the route of administration selected, the compounds of the present disclosure, which may be used in a suitable hydrated form, and/or the pharmaceutical compositions of the present disclosure, are formulated into a pharmaceutically acceptable dosage form such as described below or by other conventional methods known to those of skill in the art.

The amount and concentration of compounds of the present disclosure, e.g., a flavonoid (e.g., a compound of Table 1) or a xanthone (e.g., a compound of Table 2) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present disclosure provides pharmaceutically acceptable compositions comprising a therapeutically effective amount or prophylactically effective amount of a flavonoid (e.g., a compound of Table 1) or a xanthone (e.g., a compound of Table 2), formulated together with one or more pharmaceutically acceptable carriers (additives) and/or diluents. As described in detail below, the pharmaceutical compositions of the present disclosure may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth, (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water, (18) isotonic saline; (19) Ringer's solution; (20) ethyl alcohol; (21) phosphate buffer solutions, (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present disclosure. These salts can be prepared in situ during the final isolation and purification of the compounds of the disclosure, or by separately reacting a purified compound of the disclosure in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like (see, for example, Berge et al. (1977) “Pharmaceutical Salts”, J. Pharm. Sci. 66:1-19).

In other cases, the compounds of the present disclosure may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the compound of the present disclosure (e.g. of a flavonoid (e.g., a compound of Table 1) or a xanthone (e.g., a compound of Table 2)). These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present disclosure may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to of a flavonoid (e.g., a compound of Table 1) or a xanthone (e.g., a compound of Table 2), a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising one or more of a flavonoid (e.g., a compound of Table 1) or a xanthone (e.g., a compound of Table 2) or a pharmaceutically acceptable salt thereof.

Formulations of the present disclosure include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this disclosure suitable for parenteral administration comprise compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some embodiments, a compound of the present disclosure, such as a flavonoid (e.g., a compound of Table 1), is provided as a composition in combination with a xanthone (e.g., a compound of Table 2). For example, a flavonoid (e.g., a compound of Table 1) may be prepared as a fixed dose composition in combination with or the xanthone (e.g., a compound of Table 2). The fixed dose composition may be formulated for oral administration, e.g., as a solid dosage form or a liquid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

The combination therapy described herein may involve formulation of the component agents for different routes of administration or for the same route of administration. For example, both the flavonoid and xanthone may be formulated for oral administration. In another embodiment, the flavonoid is formulated for oral administration and the xanthone is formulated for parenteral administration. In another embodiment, the flavonoid is formulated for parenteral administration and the xanthone is formulated for oral administration. In an embodiment, the flavonoid and xanthone are formulated as a fixed dose combination (e.g., as a liquid dosage form or solid dosage form, e.g., a capsule or tablet). In some embodiments, the flavonoid and xanthone are formulated as a fixed dose combination (e.g., as a liquid dosage form or solid dosage form, e.g., a capsule or tablet) for oral administration.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin. In some cases, in order to prolong the effect of a compound of the present disclosure (e.g., a flavonoid and xanthone, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present disclosure is accomplished by dissolving or suspending compound in an oil vehicle.

In some embodiments, it may be advantageous to administer flavonoid or xanthone of the present disclosure in a sustained fashion. It will be appreciated that any formulation that provides a sustained absorption profile may be used. In certain embodiments, sustained absorption may be achieved by combining a compound of the present disclosure with other pharmaceutically acceptable ingredients, diluents, or carriers that slow its release properties into systemic circulation.

### Routes of Administration

The flavonoids and xanthones described herein, as well as other agents and related compositions thereof used in the methods described herein may be administered to a subject in a variety of forms depending on the selected route of administration, as will be understood by those skilled in the art. Exemplary routes of administration of the compositions used in the methods described herein include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrasternal, intraperitoneal, subcutaneous, intramuscular, transepithelial, nasal, intrapulmonary, intrathecal, rectal, and topical modes of administration. Parenteral administration may be by continuous infusion over a selected period of time.

In certain embodiments of the disclosure, the compositions described herein comprising a flavonoid and xanthone are administered orally. In other embodiments of the disclosure, the compositions described herein comprising a flavonoid and xanthone are administered systemically. In other embodiments of the disclosure, the compositions described herein comprising a flavonoid and xanthone are administered topically. Tn certain embodiments of the disclosure, the compositions described herein comprising a flavonoid are administered orally. In certain embodiments of the disclosure, the compositions described herein comprising a xanthone are administered orally. In other embodiments of the disclosure, the compositions described herein comprising a flavonoid are administered systemically. In other embodiments of the disclosure, the compositions described herein comprising a xanthone are administered systemically. In certain embodiments of the disclosure, the compositions described herein comprising a flavonoid are administered topically. In certain embodiments of the disclosure, the compositions described herein comprising a xanthone are administered topically. In other embodiments of the disclosure, the compositions described herein comprising a flavonoid are administered intravenously. In other embodiments of the disclosure, the compositions described herein comprising a xanthone are administered intravenously.

In an embodiment, the compositions described herein comprising luteolin is administered orally in combination with gamma-mangostin. In an embodiment, the compositions described herein comprising luteolin is administered orally prior to or after oral administration of a gamma-mangostin. In other embodiments of the disclosure, the compositions described herein comprising luteolin is administered topically (e.g., dermally). In an embodiment, the compositions described herein comprising luteolin is administered topically in combination with gamma-mangostin. In an embodiment, the compositions described herein comprising luteolin is administered systemically prior to or after administration of gamma-mangostin.

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the mute of administration will depend on whether a local or systemic effect is to be achieved. For example, for local effects, the composition can be formulated for topical administration and applied directly where its action is desired. For systemic, long-term effects, the composition can be formulated for enteral administration and given via the digestive tract. For systemic, immediate and/or short-term effects, the composition can be formulated for parenteral administration and given by routes other than through the digestive tract.

### Dosages

The compositions of the flavonoids and xanthones as described herein may be formulated into acceptable dosage forms by conventional methods known to those of skill in the art. Actual dosage levels of the active ingredients in the compositions of the present disclosure (e.g., a flavonoid or a xanthone, e.g., luteolin or gamma-mangostin) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present disclosure employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the flavonoids and/or xanthones of the disclosure employed in the composition at levels lower than that required in order to achieve the desired therapeutic effect and gradually increase the dosage until the desired effect is achieved. In general, a suitable daily dose of a composition of the present disclosure will be that amount of the substance which is the lowest dose effective to produce a therapeutic effect. Such an effective dose will generally depend upon the factors described herein. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or via injection) to a subject afflicted with a disease or disorder described herein (e.g., a inflammation or an inflammatory disease or disorder). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally, topically, or systemically) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The flavonoid and/or xanthone concentration can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

In some embodiments, the dosage of a flavonoid (e.g., a compound of Table 1) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of a flavonoid (e.g., a compound of Table 1) between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of a flavonoid (e.g., a compound of Table 1) is about 0.1 mg to about 5 mg (e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg). In some embodiments, the dosage of a flavonoid (e.g., a compound of Table 1) is between about 0.01 mg/kg to about 10 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). In some embodiments, the dosage of a flavonoid (e.g., a compound of Table 1) is between about 0.1 mg/kg to about 5 mg/kg (e.g., about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, or about 5 mg/kg).

In some embodiments, a course of a flavonoid (e.g., a compound of Table 1) is between about 1 day to about 24 weeks. In some embodiments, the course of a flavonoid (e.g., a compound of Table 1) is administered at least weekly (e.g., once a week, twice a week, three times a week, four times a week, five times a week, six times a week, 7 times a week) throughout a course of treatment. In some embodiments, the course of a flavonoid (e.g., a compound of Table 1) is administered daily throughout a course of treatment.

In some embodiments, the dosage of a xanthone (e.g., a compound of Table 2) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of a xanthone (e.g., a compound of Table 2) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of a xanthone (e.g., a compound of Table 2) is about 0.1 mg to about 5 mg (e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg). In some embodiments, the dosage of a xanthone (e.g., a compound of Table 2) is between about 0.01 mg/kg to about 10 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). In some embodiments, the dosage of a xanthone (e.g., a compound of Table 2) is between about 0.1 mg/kg to about 5 mg/kg (e.g., about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, or about 5 mg/kg).

In some embodiments, a course of a xanthone (e.g., a compound of Table 2) is between about 1 day to about 24 weeks. In some embodiments, the course of a xanthone (e.g., a compound of Table 2) is administered at least weekly (e.g., once a week, twice a week, three times a week, four times a week, five times a week, six times a week, 7 times a week) throughout a course of treatment. In some embodiments, the course of a xanthone (e.g., a compound of Table 2) is administered daily throughout a course of treatment.

### Patient Selection and Monitoring

The methods of the present disclosure described herein entail administration of a combination of a flavonoid and a xanthone. Accordingly, a patient and/or subject can be selected for treatment using a flavonoid and a xanthone for the treatment of an inflammatory disease or disorder by first evaluating the patient and/or subject to determine whether the subject has an inflammatory disease or disorder. A subject can be evaluated as having an inflammatory disease or disorder using methods known in the art. The subject can also be monitored, for example, subsequent to administration of a compound described herein (e.g., a flavonoid and a xanthone) or a pharmaceutically acceptable salt thereof.

In some embodiments, the subject is a mammal. In some embodiments, the subject is a human. In some embodiments, the subject is an adult. In some embodiments, the subject has an acute form of inflammation or an inflammatory disease or disorder. In some embodiments, the subject has a chronic form of inflammation or an inflammatory disease or disorder. In some embodiments, the subject has been diagnosed with an inflammatory disease or disorder.

In some embodiments, the subject is treatment naive. In some embodiments, the subject has previously been treated for inflammation or an inflammatory disease or disorder. For example, the subject may have received an immune therapy. In some embodiments, the subject is suffering from an inflammatory disease or disorder. In some embodiments, the subject has been treated with an agent other than a flavonoid or xanthone described herein and is suffering from a relapsed inflammatory disease or disorder.

In some embodiments, the subject has a co-morbidity, such as heart disease, coronary artery disease, a cardiomyopathy, diabetes, obesity, high blood pressure, cancer, cerebrovascular disease, chronic kidney disease, chronic liver disease, cystic fibrosis, an immunodeficiency, and tuberculosis.

In some embodiments, the methods described herein further comprise analyzing or receiving analysis of a biopsy specimen from the subject at least once prior to the end of treatment. In some embodiments, the biopsy specimen is analyzed for the levels of a cytokine, antibody, or other hypersensitivity marker.

### Additional Agents

In some embodiments, additional therapeutic agents may be administered with compositions of the present disclosure for the treatment of a inflammation or an inflammatory disease or disorder, or any symptom or associated condition thereof. When combination therapy is employed, the additional therapeutic agent(s) can be administered as a separate formulation or may be combined with any of the compositions described herein.

For example, any of the methods described herein may further comprise the administration of a therapeutically effective amount of an additional agent in conjunction with a flavonoid or a xanthone. Exemplary additional agents include an immune therapy, a vaccine, an anti-inflammatory agent, a pain reliever, a mucolytic agent, a cancer therapy, a xanthone, an antifungal agent, an antibacterial agent, a bronchodilator, or a vasodilator.

In some embodiments, the additional agent is an anti-inflammatory agent. For example, the anti-inflammatory agent may be an angiotensin-converting enzyme 2 (ACE-2) inhibitor (e.g., lisinopril, benazepril, captopril, enalapril, fosinopril, moexipril, perindopril, or quinapril), a corticosteroid (e.g., cortisone, prednisone, prednisolone, methylprednisolone, dexamethasone, betamethasone, or hydrocortisone) or a non-steroidal anti-inflammatory drug (NSAID) (e.g., ibuprofen, naproxen, diclofenac, celecoxib, mefenamic acid, etoricoxib, or indomethacin).

In some embodiments, the additional agent is a cancer therapy. In some embodiments, the cancer therapy agent is selected from methotrexate, 5-fluorouracil, doxorubicin, vincristine, bleomycin, vinblastine, dacarbazine, toposide, cisplatin, epirubicin, and sorafenib tosylate.

The compositions and methods described herein can comprise an immunomodulator. In one embodiment, the immunomodulator is an anti-inflammatory agent described herein, e.g., for treating or preventing a disease or disorder, e.g., a cancer or a fibrotic disorder described herein. The composition and method can include one, two, three or more anti-inflammatory agents, alone or in combination with one or more therapeutic agents described herein (e.g., an AHCM agent, a microenvironment modulator, an immune-checkpoint inhibitor, or an additional therapy, e.g., a cancer or anti-fibrotic therapy).

In one embodiment, the anti-inflammatory agent is an agent that blocks, inhibits, or reduces inflammation or signaling from an inflammatory signaling pathway. In one embodiment, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., TNF-α, TNF-β, TNF-RI, TNF-RII; CD23, CD30, CD40L, CXCL-1, EGF, G-CSF, GDNF, PDGF-BB, RANTES/CCL5, IKK, NF-kB, TLR2, TLR3, TLR4, TL5, TLR6, TLR7, TLR8, TLR8, TLR9, and/or any cognate receptors thereof.

In one embodiment, the anti-inflammatory agent is an IL-1 or IL-1 receptor antagonist, such as anakinra (KINIRET®), rilonacept, or canakinumab.

In one embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061.

In one embodiment, the anti-inflammatory agent is a TNF-a antagonist, e.g., an anti-TNFa antibody, such as infliximab (REMICADE®), golimumab (SIMPONI®), adalimumab (HUMIRA®), certolizumab pegol (CIMZIA®) or etanercept.

In one embodiment, the anti-inflammatory agent is a corticosteroid. Exemplary corticosteroids include, but are not limited to, cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®).

In another embodiment, the anti-inflammatory agent is a non-steroidal anti-inflammatory drug (NSAID). Exemplary anti-inflammatory agents (e.g., NSAIDs) include, but are not limited to, aspirin, ibuprofen, naproxen, celecoxib, diclofenac, diflunisal, etodolac, fenoprofen, flurbiprofen, ketoprofen, ketorolac, mefenamic acid, meloxicam, nabumetone, oxaprozin, piroxicam, sulindac, and tolmetin. In an embodiment, the anti-inflammatory agent is an immune selective anti-inflammatory derivative (ImSAID).

Administration in combination can proceed by any technique apparent to those of skill in the an including, for example, separate, sequential, concurrent, and alternating administration.

### ENUMERATED EMBODIMENTS

1. A method of reducing the level of an inflammatory cytokine in a cell or subject, wherein the method comprises administering to the cell or subject a combination of:
(i) a flavonoid; and
(ii) a xanthone,
thereby reducing the level of the inflammatory cytokine in the cell or subject.

2. The method of embodiment 1, wherein the subject has been diagnosed with dermatitis.

3. The method of any one of embodiments 1-2, wherein the inflammatory cytokine is selected from IL-6, IL-1b, and TNF-a.

4. The method of any one of embodiments 1-3, wherein the inflammatory cytokine is IL-6.

5. The method of any one of embodiments 1-3, wherein the inflammatory cytokine is IL-1b.

6. The method of any one of embodiments 1-3, wherein the inflammatory cytokine is TNF-a.

7. The method of any one of embodiments 1-6, wherein following administration, the subject has a reduced level of an inflammatory cytokine.

8. The method of any one of embodiments 1-7, wherein the level of the inflammatory cytokine is reduced by about 10%, 25%, 50%, 75%, 90%, 95%, or more relative to a reference standard.

9. The method of any one of embodiments 1-8, wherein the level of the inflammatory cytokine is reduced by about 10% relative to a reference standard.

10. The method of any one of embodiments 1-8, wherein the level of the inflammatory cytokine is reduced by about 25% relative to a reference standard.

11. The method of any one of embodiments 1-8, wherein the level of the inflammatory cytokine is reduced by about 50% relative to a reference standard.

12. The method of any one of embodiments 1-8, wherein the level of the inflammatory cytokine is reduced by about 75% relative to a reference standard.

13. The method of any one of embodiments 1-8, wherein the level of the inflammatory cytokine is reduced by about 90% relative to a reference standard.

14. The method of any one of embodiments 1-8, wherein the level of the inflammatory cytokine is reduced by about 95% relative to a reference standard.

15. A method of increasing the level of an anti-inflammatory cytokine in a cell or subject, wherein the method comprises administering to the cell or subject a combination of:
(i) a flavonoid; and
(ii) a xanthone,
thereby increasing the level of an anti-inflammatory cytokine in the cell or subject.

16. The method of embodiment 15, wherein the anti-inflammatory cytokine is selected from IL-4 and IL-10.

17. The method of any one of embodiments 15-16, wherein the anti-inflammatory cytokine is IL-4.

18. The method of any one of embodiments 15-16, wherein the anti-inflammatory cytokine is IL-10.

19. The method of any one of embodiments 15-18, wherein the level of the anti-inflammatory cytokine is increased by about 5%, 10%, 25%, 50%, 75%, 90%, 95%, or more relative to a reference standard.

20. The method of any one of embodiments 15-19, wherein the level of the anti-inflammatory cytokine is increased by about 5% or more relative to a reference standard.

21. The method of any one of embodiments 15-19, wherein the level of the anti-inflammatory cytokine is increased by about 10% or more relative to a reference standard.

22. The method of any one of embodiments 15-19, wherein the level of the anti-inflammatory cytokine is increased by about 25% or more relative to a reference standard.

23. The method of any one of embodiments 15-19, wherein the level of the anti-inflammatory cytokine is increased by about 50% or more relative to a reference standard.

24. The method of any one of embodiments 15-19, wherein the level of the anti-inflammatory cytokine is increased by about 75% or more relative to a reference standard.

25. The method of any one of embodiments 15-19, wherein the level of the anti-inflammatory cytokine is increased by about 90% or more relative to a reference standard.

26. The method of any one of embodiments 15-19, wherein the level of the anti-inflammatory cytokine is increased by about 95% or more relative to a reference standard.

27. A method of reducing the level of a hypersensitivity marker in a cell or subject, wherein the method comprises administering to the cell or subject a combination of:
(i) a flavonoid; and
(ii) a xanthone,
thereby reducing the level of a hypersensitivity marker in the cell or subject.

28. The method of embodiment 27, wherein the hypersensitivity marker is selected from IL-13, IL-14, INF-g, and serum IgE.

29. The method of any one of embodiments 27-28, wherein the hypersensitivity marker is IL-13.

30. The method of any one of embodiments 27-28, wherein the hypersensitivity marker is IL-14.

31. The method of any one of embodiments 27-28, wherein the hypersensitivity marker is INF-g.

32. The method of any one of embodiments 27-28, wherein the hypersensitivity marker is serum IgE.

33. The method of any one of embodiments 27-32, wherein the level of the hypersensitivity marker is reduced by about 10%, 25%, 50%, 75%, 90%, 95%, or more relative to a reference standard.

34. The method of any one of embodiments 27-33, wherein the level of the hypersensitivity marker is reduced by about 10% or more relative to a reference standard.

35. The method of any one of embodiments 27-33, wherein the level of the hypersensitivity marker is reduced by about 25% or more relative to a reference standard.

36. The method of any one of embodiments 27-33, wherein the level of the hypersensitivity marker is reduced by about 50% or more relative to a reference standard.

37. The method of any one of embodiments 27-33, wherein the level of the hypersensitivity marker is reduced by about 75% or more relative to a reference standard.

38. The method of any one of embodiments 27-33, wherein the level of the hypersensitivity marker is reduced by about 90% or more relative to a reference standard.

39. The method of any one of embodiments 27-33, wherein the level of the hypersensitivity marker is reduced by about 95% or more relative to a reference standard.

40. A method of inducing expression of an anti-inflammatory macrophage in a cell or subject, wherein the method comprises administering to the cell or subject a combination of:
(i) a flavonoid; and
(ii) a xanthone,
thereby inducing expression of the anti-inflammatory macrophage in the cell or subject.

41. The method of embodiment 40, wherein the anti-inflammatory macrophage is an M2 macrophage.

42. The method of any one of embodiments 40-41, wherein the M2 macrophages secrete an anti-inflammatory cytokine.

43. The method of embodiment 42, wherein the inflammatory cytokine comprises IL-10.

44. A method of improving skin health in a cell or subject, wherein the method comprises administering to the cell or subject a combination of:
(i) a flavonoid; and
(ii) a xanthone,
thereby improving skin health in the cell or subject.

45. A method of reducing inflammation in a cell or subject, wherein the method comprises administering to the cell or subject a combination of:
(i) a flavonoid; and
(ii) a xanthone,
thereby reducing inflammation in the cell or subject.

46. The method of embodiment 45, wherein the inflammation is dermal inflammation or epithelial cell inflammation.

47. The method of any one of embodiments 45-46, wherein the inflammation is dermal inflammation.

48. The method of any one of embodiments 45-46, wherein the inflammation is epithelial cell inflammation.

49. A method of treating a dermatological condition in a cell or subject, wherein the method comprises administering to the cell or subject a combination of:
(i) a flavonoid; and
(ii) a xanthone,
thereby treating the dermatological condition in a cell or subject.

50. The method of embodiment 16, wherein the dermatological condition is selected from atopic dermatitis, psoriasis, and dermal hypersensitivity.

51. The method of any one of embodiments 49-50, wherein the dermatological condition is atopic dermatitis.

52. The method of any one of embodiments 49-50, wherein the dermatological condition is psoriasis.

53. The method of any one of embodiments 49-50, wherein the dermatological condition is dermal hypersensitivity.

54. A method of treating topical inflammation in a subject, wherein the method comprises administering to the cell or subject a combination of:
(i) a flavonoid; and
(ii) a xanthone,
thereby treating topical inflammation in a subject.

55. The method of embodiment 54, wherein the topical inflammation comprises hives or a rash.

56. The method of any one of embodiments 54-55, wherein the topical inflammation comprises hives.

57. The method of any one of embodiments 54-55, wherein the topical inflammation comprises a rash.

58. A method of treating an inflammatory disease or disorder in a cell or subject, wherein the method comprises administering to the cell or subject a combination of:
(i) a flavonoid; and
(ii) a xanthone,
thereby treating an inflammatory disease or disorder in a cell or subject.

59. The method of embodiment 58, wherein the inflammatory disease or disorder condition comprises rheumatoid arthritis.

60. A method of reducing a symptom of inflammation or an inflammatory disease or disorder in a subject, wherein the method comprises administering to the cell or subject a combination of:
(i) a flavonoid; and
(ii) a xanthone,
thereby reducing the symptom of inflammation or an inflammatory disease or disorder in a subject.

61. The method of embodiment 60, wherein the symptom comprises swelling, irritation, redness, itching, hives, or sneezing.

62. The method of any one of embodiments 60-61, wherein the symptom comprises swelling.

63. The method of any one of embodiments 60-61, wherein the symptom comprises irritation.

64. The method of any one of embodiments 60-61, wherein the symptom comprises redness.

65. The method of any one of embodiments 60-61, wherein the symptom comprises itching.

66. The method of any one of embodiments 60-61, wherein the symptom comprises hives.

67. The method of any one of embodiments 60-61, wherein the symptom comprises sneezing.

68. The method of any one of embodiments 1-67, wherein one of the flavonoid and the xanthone comprises an AHR agonist, an NRF2 agonist, or a PPARg agonist.

69. The method of any one of embodiments 1-68, wherein one of the flavonoid and the xanthone comprises an AHR agonist.

70. The method of any one of embodiments 1-68, wherein one of the flavonoid and the xanthone comprises an NRF2 agonist.

71. The method of any one of embodiments 1-68, wherein one of the flavonoid and the xanthone comprises an PPARg agonist.

72. The method of any one of embodiments 1-71, wherein each of the flavonoid and the xanthone independently comprises an AHR agonist, an NRF2 agonist, or a PPARg agonist.

73. The method of any one of embodiments 1-72, wherein each of the flavonoid and the xanthone independently comprises an AHR agonist.

74. The method of any one of embodiments 1-72, wherein each of the flavonoid and the xanthone independently comprises an NRF2 agonist.

75. The method of any one of embodiments 1-72, wherein each of the flavonoid and the xanthone independently comprises an PPARg agonist.

76. The method of any one of embodiments 1-75, wherein the xanthone has the structure of Formula (B):

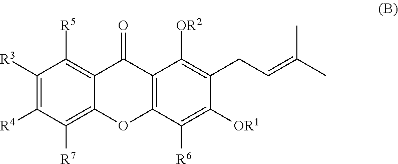

or a pharmaceutically acceptable salt thereof, wherein:
each of R¹ and R² is independently hydrogen or C₁-C₆ alkyl;
each of R³, R⁴, and R⁷ is independently hydrogen, —OR^{A}, C₁-C₆ alkyl, C₂-C₆ alkenyl, C₂-C₆alkynyl, or C₂-C₁₀ alkenyl;
each of R⁵ and R⁶ is independently hydrogen, C₁-C₆ alkyl, C₂-C₆ alkenyl, C₂-C₆ alkynyl, —OR^{A} or C₂-C₁₀ alkenyl; and
R^{A} is hydrogen, C₁-C₆ alkyl, or cycloalkyl.

77. The method of any one of embodiments 1-75, wherein the xanthone has the structure of Formula (B-i):

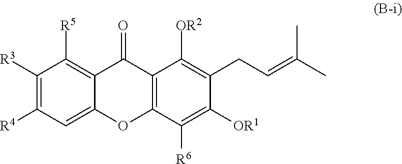

or a pharmaceutically acceptable salt thereof, wherein:
each of R¹ and R² is independently hydrogen or C₁-C₆ alkyl;
each of R³ and R⁴ is independently hydrogen or —OR^{A};
each of R⁵ and R⁶ is independently hydrogen, C₁-C₆ alkyl, C₂-C₆ alkenyl, C₂-C₆ alkynyl, or —OR^{A}; and
R^{A} is hydrogen, C₁-C₆ alkyl, or cycloalkyl.

78. The method of embodiment 77, wherein R¹ is hydrogen.

79. The method of any one of embodiments 77-78, wherein R² is hydrogen.

80. The method of any one of embodiments 77-79, wherein R³ is —OR.

81. The method of any one of embodiments 77-79, wherein R³ is —OH.

82. The method of any one of embodiments 77-81, wherein R⁴ is —OR^{A}.

83. The method of any one of embodiments 77-81, wherein R⁴ is —OH.

84. The method of any one of embodiments 77-83, wherein R⁵ is C₂-C₆ alkenyl.

85. The method of any one of embodiments 77-83, wherein R is —CH₂CH═C(CH₃)₂.

86. The method of any one of embodiments 77-85, wherein R⁶ is hydrogen.

87. The method of any one of embodiments 77-86, wherein each of R¹ and R² is hydrogen; each of R³ and R⁴ is —OR^{A}; R⁵ is C₂-C₆ alkenyl; and R⁶ is hydrogen.

88. The method of any one of embodiments 1-75, wherein the xanthone has the structure of Formula (B-ii):

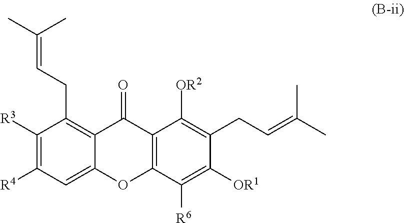

or a pharmaceutically acceptable salt thereof, wherein:
each of R¹ and R² is independently hydrogen or C₁-C₆ alkyl;
each of R³ and R⁴ is independently hydrogen or —OR^{A};
R⁶ is hydrogen, C₁-C₆ alkyl, C₂-C₆ alkenyl, C₂-C₆ alkynyl, or —OR^{A}; and
R^{A} is hydrogen, C₁-C₆ alkyl, or cycloalkyl.

89. The method of embodiment 88, wherein each of R¹ and R² is hydrogen; each of R³ and R⁴ is —OR^{A}; and R⁶ is hydrogen.

90. The method of any one of embodiments 1-75, wherein the xanthone is a compound of Formula (B-iii):

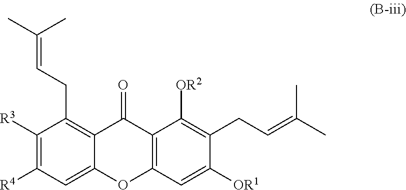

or a pharmaceutically acceptable salt thereof, wherein:
each of R¹ and R² is independently hydrogen or C₁-C₆ alkyl;
each of R³ and R⁴ is independently hydrogen or —OR^{A}; and
R^{A} is hydrogen, C₁-C₆ alkyl, or cycloalkyl.

91. The method of embodiment 90, wherein each of R¹ and R² is hydrogen; and each of R³ and R⁴ is —OR^{A}.

92. The method of embodiment 90, wherein each of R¹ and R² is hydrogen; R³ is —OCH₃, and R⁴ is —OH.

93. The method of any one of embodiments 1-92, wherein the xanthone is selected from alpha-mangostin, beta-mangostin, gamma-mangostin, 8-deoxygartanin, garcinone E, gartanin, 1,7-dihydroxy-3-methoxy-2-prenylxanthone, 1,7-dihydroxy-3,6-dimethoxy-2,8-diprenylxanthone, morusignin B, dulxanthone B, 1,5-dihydroxy-3-methoxy-2-prenylxanthone, 1,4,6-trihydroxy-5-methoxy-7-prenylxanthone, nigrolineaxanthone E, l-Hydroxy-3,5-dimethoxy-2,4-diprenylxanthone, gerontoxanthone I, 6-deoxy-gamma-mangostin, cowanin, dulcisxanthone B, cowaxanthone B, 1,3,7-trihydroxy-2,4-diisoprenylxanthone, cratoxyarborenone B, 1,3,5-trihydroxy-2-prenyl-9H-xanthene-9-one, 1,3,7-trihydroxy-2-prenylxanthone, doitunggarcinone C, cudratricusxanthone K, 1-hydroxy-3,5-dimethoxy-2-prenylxanthone, 1,8-dihydroxy-3,5-dimethoxy-2-prenylxanthone, or a pharmaceutically acceptable salt thereof.

94. The method of any one of embodiments 1-93, wherein the xanthone is selected from alpha-mangostin, beta-mangostin, gamma-mangostin, 8-deoxygartanin, garcinone E, gartanin, or a pharmaceutically acceptable salt thereof.

95. The method of any one of embodiments 1-94, wherein the xanthone is selected from alpha-mangostin, beta-mangostin, gamma-mangostin, and 8-deoxygartanin, or a pharmaceutically acceptable salt thereof.

96. The method of any one of embodiments 1-95, wherein the xanthone is selected from alpha-mangostin, gamma-mangostin, or a pharmaceutically acceptable salt thereof.

97. The method of any one of embodiments 1-96, wherein the xanthone is gamma-mangostin or a pharmaceutically acceptable salt thereof.

98. The method of any one of embodiments 1-97, wherein the flavonoid has the structure of Formula (A):

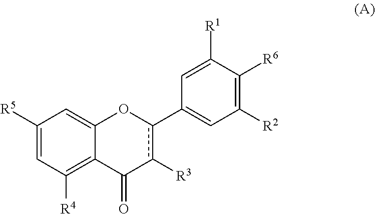

or a pharmaceutically acceptable salt thereof, wherein:
each of R¹, R², R³, R⁴, R⁵, and R⁶ is independently hydrogen or —OR^{A};
R^{A} is hydrogen, C₁-C₆ alkyl, or cycloalkyl; and
“ [symbol] ” is a single or double bond.

99. The method of any one of embodiments 1-97, wherein the flavonoid has the structure of Formula (A-i):

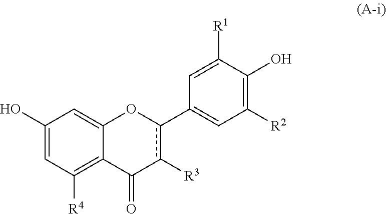

or a pharmaceutically acceptable salt thereof, wherein:
each of R¹ and R² is independently hydrogen or OR^{A};
R³ is hydrogen or OR^{A};
R⁴ is hydrogen or —OR^{A};
R^{A} is hydrogen, C₁-C₆ alkyl, or cycloalkyl; and
“ [symbol] ” is a single or double bond.

100. The method of any one of embodiments 1-97, wherein the flavonoid has the structure of Formula (A-ii);

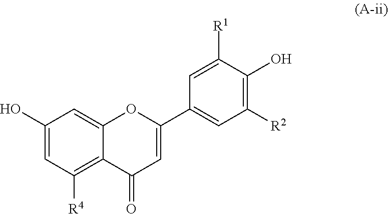

or a pharmaceutically acceptable salt thereof, wherein:
each of R¹ and R² is independently hydrogen or —OR^{A};
R⁴ is hydrogen or —OR^{A}; and
R^{A} is hydrogen, C₁-C₆ alkyl, or cycloalkyl.

101. The method of embodiment 100, wherein R¹ is —OR^{A}; R² is hydrogen; and R⁴ is —OR^{A}.

102. The method of embodiment 100, wherein R is —OH; R² is hydrogen; and R⁴ is —OH.

103. The method of any one of embodiments 1-97, wherein the flavonoid has the structure of Formula (A-iii):

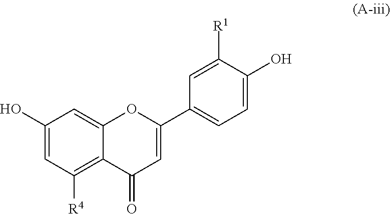

or a pharmaceutically acceptable salt thereof, wherein:
each of R¹ is hydrogen or —OR^{A};
R⁴ is hydrogen or —OR^{A}; and
R^{A} is hydrogen, C₁-C₆ alkyl, or cycloalkyl.

104. The method of embodiment 103, wherein R¹ is —OR^{A} and R⁴ is —OR^{A}.

105. The method of embodiment 103, wherein R¹ is —OH and R⁴ is —OH.

106. The method of any one embodiments 1-105, wherein the flavonoid is selected from quercetin, luteolin, myricetin, taxifolin, fisetin, kaempferol, apigenin, tricetin, isohamnetin, eriodictyol, dihydromyricetin, chryseriol, 7,3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin, or a pharmaceutically acceptable salt thereof.

107. The method of any one of embodiments 1-106, wherein the flavonoid is selected from quercetin, luteolin, myricetin, taxifolin, fisetin, kaempferol, or a pharmaceutically acceptable salt thereof.

108. The method of any one of embodiments 1-107, wherein the flavonoid is selected from luteolin, myricetin, taxifolin, fisetin, kaempferol, or a pharmaceutically acceptable salt thereof.

109. The method of any one of embodiments 1-108, wherein the flavonoid is quercetin, luteolin, or a pharmaceutically acceptable salt thereof.

110. The method of any one of embodiments 1-109, wherein the flavonoid is luteolin or a pharmaceutically acceptable salt thereof.

111. The method of any one of embodiments 1-110, wherein the efficacy of the combination is at least X₁-fold greater than the efficacy of the flavonoid alone at the molar amount used in the combination, wherein X₁ is 1, 1.25, 1.5, 1.75, 2, 2.5, or greater.

112. The method of any one of embodiments 1-111, wherein the efficacy of the combination is at least X₁-fold greater than the efficacy of the flavonoid alone at the molar amount used in the combination, wherein X₁ is 1 or greater.

113. The method of any one of embodiments 1-111, wherein the efficacy of the combination is at least X₁-fold greater than the efficacy of the flavonoid alone at the molar amount used in the combination, wherein X₁ is 1.25 or greater.

114. The method of any one of embodiments 1-111, wherein the efficacy of the combination is at least X₁-fold greater than the efficacy of the flavonoid alone at the molar amount used in the combination, wherein X₁ is 1.5 or greater.

115. The method of any one of embodiments 1-111, wherein the efficacy of the combination is at least X₁-fold greater than the efficacy of the flavonoid alone at the molar amount used in the combination, wherein X₁ is 1.75 or greater.

116. The method of any one of embodiments 1-111, wherein the efficacy of the combination is at least X₁-fold greater than the efficacy of the flavonoid alone at the molar amount used in the combination, wherein X₁ is 2 or greater.

117. The method of any one of embodiments 1-111, wherein the efficacy of the combination is at least X₁-fold greater than the efficacy of the flavonoid alone at the molar amount used in the combination, wherein X₁ is 2.5 or greater.

118. The method of any one of embodiments 1-117, wherein the efficacy of the combination is at least X₁-fold greater than the efficacy of the xanthone alone at the molar amount used in the combination, wherein X₁ is 1, 1.25, 1.5, 1.75, 2, 2.5, or greater.

119. The method of any one of embodiments 1-118, wherein the efficacy of the combination is at least X₁-fold greater than the efficacy of the xanthone alone at the molar amount used in the combination, wherein X₁ is 1 or greater.

120. The method of any one of embodiments 1-118, wherein the efficacy of the combination is at least X₁-fold greater than the efficacy of the xanthone alone at the molar amount used in the combination, wherein X₁ is 1.25 or greater.

121. The method of any one of embodiments 1-118, wherein the efficacy of the combination is at least X₁-fold greater than the efficacy of the xanthone alone at the molar amount used in the combination, wherein X₁ is 1.5 or greater.

122. The method of any one of embodiments 1-118, wherein the efficacy of the combination is at least X₁-fold greater than the efficacy of the xanthone alone at the molar amount used in the combination, wherein X₁ is 1.75 or greater.

123. The method of any one of embodiments 1-118, wherein the efficacy of the combination is at least X₁-fold greater than the efficacy of the xanthone alone at the molar amount used in the combination, wherein X₁ is 2 or greater.

124. The method of any one of embodiments 1-118, wherein the efficacy of the combination is at least X₁-fold greater than the efficacy of the xanthone alone at the molar amount used in the combination, wherein X₁ is 2.5 or greater.

125. The method of any one of embodiments 1-124, wherein the xanthone is gamma-mangostin and the flavonoid is luteolin.

126. The method of any one of embodiments 1-125, wherein the xanthone is prepared synthetically.

127. The method of any one of embodiments 1-126, wherein the flavonoid is prepared synthetically.

128. The method of any one of embodiments 1-127, wherein the xanthone and/or the flavonoid is extracted from a natural source (e.g., a plant).

129. The method of any one of embodiments 1-128, wherein the flavonoid is a compound selected from Table 1 or a pharmaceutically acceptable salt thereof.

130. The method of embodiment 129, wherein the flavonoid is substantially pure.

131. The method of any one of embodiments 1-130, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of another compound.

132. The method of embodiment 131, wherein the pharmaceutical composition comprises less than about 50% of another compound.

133. The method of embodiment 131, wherein the pharmaceutical composition comprises less than about 60% of another compound.

134. The method of embodiment 131, wherein the pharmaceutical composition comprises less than about 65% of another compound.

135. The method of embodiment 131, wherein the pharmaceutical composition comprises less than about 70% of another compound.

136. The method of embodiment 131, wherein the pharmaceutical composition comprises less than about 75% of another compound.

137. The method of embodiment 131, wherein the pharmaceutical composition comprises less than about 80% of another compound.

138. The method of embodiment 131, wherein the pharmaceutical composition comprises less than about 85% of another compound.

139. The method of embodiment 131, wherein the pharmaceutical composition comprises less than about 90% of another compound.

140. The method of embodiment 131, wherein the pharmaceutical composition comprises less than about 95% of another compound.

141. The method of embodiment 131, wherein the pharmaceutical composition comprises less than about 99% of another compound.

142. The method of embodiment 131, wherein the pharmaceutical composition comprises less than about 99.9% of another compound.

143. The method of any one of embodiments 1-142, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of another flavonoid, such as a flavonoid listed in Table 1 or a pharmaceutically acceptable salt thereof.

144. The method of embodiment 143, wherein the pharmaceutical composition comprises less than about 50% of another flavonoid.

145. The method of embodiment 143, wherein the pharmaceutical composition comprises less than about 60% of another flavonoid.

146. The method of embodiment 143, wherein the pharmaceutical composition comprises less than about 65% of another flavonoid.

147. The method of embodiment 143, wherein the pharmaceutical composition comprises less than about 70% of another flavonoid.

148. The method of embodiment 143, wherein the pharmaceutical composition comprises less than about 75% of another flavonoid.

149. The method of embodiment 143, wherein the pharmaceutical composition comprises less than about 80% of another flavonoid.

150. The method of embodiment 143, wherein the pharmaceutical composition comprises less than about 85% of another flavonoid.

151. The method of embodiment 143, wherein the pharmaceutical composition comprises less than about 90% of another flavonoid.

152. The method of embodiment 143, wherein the pharmaceutical composition comprises less than about 95% of another flavonoid.

153. The method of embodiment 143, wherein the pharmaceutical composition comprises less than about 99% of another flavonoid.

154. The method of embodiment 143, wherein the pharmaceutical composition comprises less than about 99.9% of another flavonoid.

155. The method of any one of embodiments 1-154, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of a plant-derived substance.

156. The method of embodiment 155, wherein the pharmaceutical composition comprises less than about 50% of a plant-derived substance.

157. The method of embodiment 155, wherein the pharmaceutical composition comprises less than about 55% of a plant-derived substance.

158. The method of embodiment 155, wherein the pharmaceutical composition comprises less than about 60% of a plant-derived substance.

159. The method of embodiment 155, wherein the pharmaceutical composition comprises less than about 65% of a plant-derived substance.

160. The method of embodiment 155, wherein the pharmaceutical composition comprises less than about 70% of a plant-derived substance.

161. The method of embodiment 155, wherein the pharmaceutical composition comprises less than about 75% of a plant-derived substance.

162. The method of embodiment 155, wherein the pharmaceutical composition comprises less than about 80% of a plant-derived substance.

163. The method of embodiment 155, wherein the pharmaceutical composition comprises less than about 85% of a plant-derived substance.

164. The method of embodiment 155, wherein the pharmaceutical composition comprises less than about 90% of a plant-derived substance.

165. The method of embodiment 155, wherein the pharmaceutical composition comprises less than about 95% of a plant-derived substance.

166. The method of embodiment 155, wherein the pharmaceutical composition comprises less than about 99% of a plant-derived substance.

167. The method of embodiment 155, wherein the pharmaceutical composition comprises less than about 99.9% of a plant-derived substance.

168. The method of any one of embodiments 1-167, wherein the flavonoid is provided in the combination in the absence of quercetin, luteolin, myricetin, taxifolin, fisetin, kaempferol, apigenin, tricetin, isohamnetin, eriodictyol, dihydromyricetin, chryseriol, 7.3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin or a pharmaceutically acceptable salt thereof.

169. The method of any one of embodiments 1-168, wherein the flavonoid is luteolin or a pharmaceutically acceptable salt thereof.

170. The method of embodiment 169, wherein the luteolin or a pharmaceutically acceptable salt thereof is provided in the combination in the absence of quercetin, luteolin, myricetin, taxifolin, fisetin, kaempferol, apigenin, tricetin, isohamnetin, eriodictyol, dihydromyricetin, chryseriol, 7,3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin, or a pharmaceutically acceptable salt thereof.

171. The method of any one of embodiments 169-170, wherein the luteolin or a pharmaceutically acceptable salt thereof is provided as a pharmaceutical composition, and the pharmaceutical composition comprises less than about 90%, 95%, 99%, or 99.9% of another compound in listed in Table 1 or a pharmaceutically acceptable salt thereof.

172. The method of embodiment 171, wherein the pharmaceutical composition comprises less than about 90% of another compound listed in Table 1 or a pharmaceutically acceptable salt thereof.

173. The method of embodiment 171, wherein the pharmaceutical composition comprises less than about 95% of another compound listed in Table 1 or a pharmaceutically acceptable salt thereof.

174. The method of embodiment 171, wherein the pharmaceutical composition comprises less than about 99% of another compound listed in Table 1 or a pharmaceutically acceptable salt thereof.

175. The method of embodiment 171, wherein the pharmaceutical composition comprises less than about 99.9% of another compound listed in Table 1 or a pharmaceutically acceptable salt thereof.

176. The method of any one of embodiments 1-175, wherein the xanthone is a compound selected from Table 1 or a pharmaceutically acceptable salt thereof.

177. The method of embodiment 176, wherein the xanthone is substantially pure.

178. The method of any one of embodiments 176-177, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of another compound.

179. The method of any one of embodiment 178, wherein the pharmaceutical composition comprises less than about 50% of another compound.

180. The method of any one of embodiment 178, wherein the pharmaceutical composition comprises less than about 55% of another compound.

181. The method of any one of embodiment 178, wherein the pharmaceutical composition comprises less than about 60% of another compound.

182. The method of any one of embodiment 178, wherein the pharmaceutical composition comprises less than about 65% of another compound.

183. The method of any one of embodiment 178, wherein the pharmaceutical composition comprises less than about 70% of another compound.

184. The method of any one of embodiment 178, wherein the pharmaceutical composition comprises less than about 75% of another compound.

185. The method of any one of embodiment 178, wherein the pharmaceutical composition comprises less than about 80% of another compound.

186. The method of any one of embodiment 178, wherein the pharmaceutical composition comprises less than about 85% of another compound.

187. The method of any one of embodiment 178, wherein the pharmaceutical composition comprises less than about 90% of another compound.

188. The method of any one of embodiment 178, wherein the pharmaceutical composition comprises less than about 95% of another compound.

189. The method of any one of embodiment 178, wherein the pharmaceutical composition comprises less than about 99% of another compound.

190. The method of any one of embodiment 178, wherein the pharmaceutical composition comprises less than about 99.9% of another compound.

191. The method of any one of embodiments 1-190, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of another xanthone, such as a xanthone listed in Table 2 or a pharmaceutically acceptable salt thereof.

192. The method of embodiment 191, wherein the pharmaceutical composition comprises less than about 50% of another xanthone.

193. The method of embodiment 191, wherein the pharmaceutical composition comprises less than about 55% of another xanthone.

194. The method of embodiment 191, wherein the pharmaceutical composition comprises less than about 60% of another xanthone.

195. The method of embodiment 191, wherein the pharmaceutical composition comprises less than about 65% of another xanthone.

196. The method of embodiment 191, wherein the pharmaceutical composition comprises less than about 70% of another xanthone.

197. The method of embodiment 191, wherein the pharmaceutical composition comprises less than about 75% of another xanthone.

198. The method of embodiment 191, wherein the pharmaceutical composition comprises less than about 80% of another xanthone.

199. The method of embodiment 191, wherein the pharmaceutical composition comprises less than about 85% of another xanthone.

200. The method of embodiment 191, wherein the pharmaceutical composition comprises less than about 90% of another xanthone.

201. The method of embodiment 191, wherein the pharmaceutical composition comprises less than about 95% of another xanthone.

202. The method of embodiment 191, wherein the pharmaceutical composition comprises less than about 99% of another xanthone.

203. The method of embodiment 191, wherein the pharmaceutical composition comprises less than about 99.9% of another xanthone.

204. The method of any one of embodiments 1-203, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of a plant-derived substance.

205. The method of embodiment 204, wherein the pharmaceutical composition comprises less than about 50% of a plant-derived substance.

206. The method of embodiment 204, wherein the pharmaceutical composition comprises less than about 55% of a plant-derived substance.

207. The method of embodiment 204, wherein the pharmaceutical composition comprises less than about 60% of a plant-derived substance.

208. The method of embodiment 204, wherein the pharmaceutical composition comprises less than about 65% of a plant-derived substance.

209. The method of embodiment 204, wherein the pharmaceutical composition comprises less than about 70% of a plant-derived substance.

210. The method of embodiment 204, wherein the pharmaceutical composition comprises less than about 75% of a plant-derived substance.

211. The method of embodiment 204, wherein the pharmaceutical composition comprises less than about 80% of a plant-derived substance.

212. The method of embodiment 204, wherein the pharmaceutical composition comprises less than about 85% of a plant-derived substance.

213. The method of embodiment 204, wherein the pharmaceutical composition comprises less than about 90% of a plant-derived substance.

214. The method of embodiment 204, wherein the pharmaceutical composition comprises less than about 95% of a plant-derived substance.

215. The method of embodiment 204, wherein the pharmaceutical composition comprises less than about 99% of a plant-derived substance.

216. The method of embodiment 204, wherein the pharmaceutical composition comprises less than about 99.9% of a plant-derived substance.

217. The method of any one of embodiments 1-216, wherein the flavonoid is provided in the combination in the absence of hyperforin, adhyperforin, or a pharmaceutically acceptable salt thereof.

218. The method of any one of embodiments 1-217, wherein the xanthone is gamma-mangostin or a pharmaceutically acceptable salt thereof.

219. The method of embodiment 218, wherein the gamma-mangostin or a pharmaceutically acceptable salt thereof is provided in the combination in the absence of alpha-mangostin, beta-mangostin, 8-deoxygartanin, garcinone E, gartanin, 1,7-dihydroxy-3-methoxy-2-prenylxanthone, 1,7-dihydroxy-3,6-dimethoxy-2,8-diprenylxanthone, morusignin B, dulxanthone B, 1,5-dihydroxy-3-methoxy-2-prenylxanthone, 1,4,6-trihydroxy-5-methoxy-7-prenylxanthone, nigrolineaxanthone E, 1-Hydroxy-3,5-dimethoxy-2,4-diprenylxanthone, gerontoxanthone I, 6-deoxy-gamma-mangostin, cowanin, dulcisxanthone B, cowaxanthone B, 1,3,7-trihydroxy-2,4-diisoprenylxanthone, cratoxyarborenone B, 1,3,5-trihydroxy-2-prenyl-9H-xanthene-9-one, 1,3,7-trihydroxy-2-prenylxanthone, doitunggarcinone C, cudratricusxanthone K, 1-hydroxy-3,5-dimethoxy-2-prenylxanthone, 1,8-dihydroxy-3,5-dimethoxy-2-prenylxanthone, or a pharmaceutically acceptable salt thereof.

220. The method of any one of embodiments 218-219, wherein the luteolin or a pharmaceutically acceptable salt thereof is provided as a pharmaceutical composition, and the pharmaceutical composition comprises less than about 90%, 95%, 99%, or 99.9% of another compound listed in Table 2 or a pharmaceutically acceptable salt thereof.

221. The method of embodiment 220, wherein the pharmaceutical composition comprises less than about 90% of another compound listed in Table 2 or a pharmaceutically acceptable salt thereof.

222. The method of embodiment 220, wherein the pharmaceutical composition comprises less than about 95% of another compound listed in Table 2 or a pharmaceutically acceptable salt thereof.

223. The method of embodiment 220, wherein the pharmaceutical composition comprises less than about 99% of another compound listed in Table 2 or a pharmaceutically acceptable salt thereof.

224. The method of embodiment 220, wherein the pharmaceutical composition comprises less than about 99.9% of another compound listed in Table 2 or a pharmaceutically acceptable salt thereof.

225. The method of any one of embodiments 1-224, wherein each of gamma-mangostin and luteolin is independently formulated as a pharmaceutical composition.

226. The method of any one of embodiments 1-225, wherein the gamma-mangostin and luteolin are formulated together as a pharmaceutical composition.

227. The method of any one of embodiments 1-226, wherein each of the gamma-mangostin and luteolin is provided concomitantly to the subject.

228. The method of embodiment 227, wherein each of the gamma-mangostin and luteolin is provided sequentially to the subject.

229. The method of embodiment 228, wherein the gamma-mangostin is provided to the subject prior to the luteolin.

230. The method of embodiment 229, wherein the luteolin is provided to the subject prior to the gamma-mangostin.

231. The method of any one of embodiments 1-230, wherein the xanthone and the flavonoid are formulated for topical, systemic, or oral administration.

232. The method of embodiment 231, wherein the xanthone and the flavonoid are formulated for topical administration.

233. The method of embodiment 232, wherein the xanthone and the flavonoid are formulated for systemic administration.

234. The method of embodiment 233, wherein the xanthone and the flavonoid are formulated for oral administration.

235. The method of any one of embodiments 1-234, wherein the flavonoid is administered at a dosage to provide a concentration of between 0.1 μM and 500 μM in the subject or a cell.

236. The method of any one of embodiments 1-235, wherein the flavonoid is administered at a dosage to provide a concentration of between 1 μM and 25 μM in the subject or a cell.

237. The method of any one of embodiments 1-236, wherein the xanthone is administered at a dosage to provide a concentration of between 0.01 μM and 25 μM in the subject or a cell.

238. The method of any one of embodiments 1-237, wherein the xanthone is administered at a dosage to provide a concentration of between 0.1 μM and 5 μM in the subject or a cell.

239. The method of any one of embodiments 1-238, wherein the ratio of the amount of flavonoid to the xanthone in the combination is between 200:1 to 1:1.

240. The method of embodiment 239, wherein the ratio of the amount of flavonoid to the xanthone in the combination is between 50:1 to 1:1.

241. The method of any one of embodiments 239-240, wherein the ratio of the amount of flavonoid to the xanthone in the combination is between 50:1 to 2:1.

242. The method of any one of embodiments 239-241, wherein the ratio of the amount of flavonoid to the xanthone in the combination is between 25:1 to 2:1.

243. The method of any one of embodiments 1-242, wherein:
(i) the flavonoid is luteolin or a pharmaceutically acceptable salt thereof;
(ii) the xanthone is gamma-mangostin or a pharmaceutically acceptable salt thereof; and
(iii) the molar amount of the flavonoid in the combination is between 20-fold and 5-fold greater than the molar amount of the xanthone.

244. The method of any one of embodiments 1-243, further comprising administration of an additional agent.

245. The method of any one of embodiments 1-244, wherein the subject is a mammal (e.g., a human.

246. The method of any one of embodiments 1-245, wherein the inflammation is present within an inflamed organ, tissue, or cell in a subject.

247. The method of embodiment 246, wherein the inflammation is present within an inflamed organ in the subject.

248. The method of embodiment 247, wherein the inflamed organ is selected from the group consisting of the skin, brain, spinal cord, eye, lung, heart, pancreas, large intestine, small intestine, stomach, liver, gall bladder, kidney, or spleen.

249. The method of embodiment 248, wherein the inflamed organ is the skin.

250. The method of embodiment 248, wherein the inflamed organ is the brain.

251. The method of embodiment 248, wherein the inflamed organ is the spinal cord.

252. The method of embodiment 248, wherein the inflamed organ is the eye.

253. The method of embodiment 248, wherein the inflamed organ is the lung.

254. The method of embodiment 248, wherein the inflamed organ is the heart.

255. The method of embodiment 248, wherein the inflamed organ is the pancreas.

256. The method of embodiment 248, wherein the inflamed organ is the large intestine.

257. The method of embodiment 248, wherein the inflamed organ is the small intestine.

258. The method of embodiment 248, wherein the inflamed organ is the stomach.

259. The method of embodiment 248, wherein the inflamed organ is the liver.

260. The method of embodiment 248, wherein the inflamed organ is the gall bladder.

261. The method of embodiment 248, wherein the inflamed organ is the kidney.

262. The method of embodiment 248, wherein the inflamed organ is the spleen.

263. The method of embodiment 246, wherein the inflammation is present within an inflamed tissue in the subject.

264. The method of embodiment 263, wherein the inflamed tissue is selected from the group consisting of lung tissue, tracheal tissue, intestinal tissue, skin tissue, pancreatic tissue, vascular tissue, mucosal tissue, kidney tissue, brain tissue, nervous tissue, or cardiac tissue.

265. The method of embodiment 264, wherein the inflamed tissue is lung tissue.

266. The method of embodiment 264, wherein the inflamed tissue is tracheal tissue.

267. The method of embodiment 264, wherein the inflamed tissue is intestinal tissue.

268. The method of embodiment 264, wherein the inflamed tissue is skin tissue.

269. The method of embodiment 264, wherein the inflamed tissue is pancreatic tissue.

270. The method of embodiment 264, wherein the inflamed tissue is vascular tissue.

271. The method of embodiment 264, wherein the inflamed tissue is mucosal tissue.

272. The method of embodiment 264, wherein the inflamed tissue is kidney tissue.

273. The method of embodiment 264, wherein the inflamed tissue is brain tissue.

274. The method of embodiment 264, wherein the inflamed tissue is nervous tissue.

275. The method of embodiment 264, wherein the inflamed tissue is cardiac tissue.

276. A composition for use in reducing the level of an inflammatory cytokine in a cell or subject, wherein the composition comprises a combination of:
(i) a flavonoid; and
(ii) a xanthone.

277. A composition for use in reducing the level of an inflammatory cytokine in a cell or subject, wherein the composition comprises a combination of:
(i) a luteolin; and
(ii) a xanthone.

278. A composition for use in reducing the level of an inflammatory cytokine in a cell or subject, wherein the composition comprises a combination of:
(i) a flavonoid; and
(ii) a gamma-mangostin.

279. A composition for use in reducing the level of an inflammatory cytokine in a cell or subject, wherein the composition comprises a combination of:
(i) a luteolin; and
(ii) a gamma-mangostin.

280. The composition for use of any one of embodiments 276-279, wherein the subject has been diagnosed with dermatitis.

281. The composition for use of any one of embodiments 276-280, wherein following administration of the composition to the subject, the subject has a reduced level of an inflammatory cytokine.

282. The composition for use of any one of embodiments 276-281, wherein the inflammatory cytokine is selected from IL-6, IL-1b, and TNA-a.

283. The composition for use of any one of embodiments 276-282, wherein the inflammatory cytokine is IL-6.

284. The composition for use of any one of embodiments 276-283, wherein the inflammatory cytokine is IL-1b.

285. The composition for use of any one of embodiments 276-284, wherein the inflammatory cytokine is TNF-a.

286. The composition for use of any one of embodiments 276-285, wherein the level of the inflammatory cytokine is reduced by about 10%, 25%, 50%, 75%, 90%, 95%, or more relative to a reference standard.

287. The composition for use of any one of embodiments 276-286, wherein the level of the inflammatory cytokine is reduced by about 10% relative to a reference standard.

288. The composition for use of any one of embodiments 276-286, wherein the level of the inflammatory cytokine is reduced by about 25% relative to a reference standard.

289. The composition for use of any one of embodiments 276-286, wherein the level of the inflammatory cytokine is reduced by about 50% relative to a reference standard.

290. The composition for use of any one of embodiments 276-286, wherein the level of the inflammatory cytokine is reduced by about 75% relative to a reference standard.

291. The composition for use of any one of embodiments 276-286, wherein the level of the inflammatory cytokine is reduced by about 90% relative to a reference standard.

292. The composition for use of any one of embodiments 276-286, wherein the level of the inflammatory cytokine is reduced by about 95% relative to a reference standard.

293. A composition for use in increasing the level of an anti-inflammatory cytokine in a subject, wherein the composition for use comprises a combination of:
(i) a flavonoid; and
(ii) a xanthone.

294. The composition for use of embodiment 293, wherein following administration of the composition to the subject, the subject has an increased level of an anti-inflammatory cytokine.

295. The composition for use of embodiment 294, wherein the anti-inflammatory cytokine is selected from IL-4 and IL-10.

296. The composition for use of any one of embodiments 293-294, wherein the anti-inflammatory cytokine is ILA.

297. The composition for use of any one of embodiments 293-294, wherein the anti-inflammatory cytokine is IL-10.

298. The composition for use of any one of embodiments 293-297, wherein the level of the anti-inflammatory cytokine is increased by about 5%, 10%, 25%, 50%, 75%, 90%, 95%, or more relative to a reference standard.

299. The composition for use of any one of embodiments 293-298, wherein the level of the anti-inflammatory cytokine is increased by about 5% or more relative to a reference standard.

300. The composition for use of any one of embodiments 293-298, wherein the level of the anti-inflammatory cytokine is increased by about 10% or more relative to a reference standard.

301. The composition for use of any one of embodiments 293-298, wherein the level of the anti-inflammatory cytokine is increased by about 25% or more relative to a reference standard.

302. The composition for use of any one of embodiments 293-298, wherein the level of the anti-inflammatory cytokine is increased by about 50% or more relative to a reference standard.

303. The composition for use of any one of embodiments 293-298, wherein the level of the anti-inflammatory cytokine is increased by about 75% or more relative to a reference standard.

304. The composition for use of any one of embodiments 293-298, wherein the level of the anti-inflammatory cytokine is increased by about 90% or more relative to a reference standard.

305. The composition for use of any one of embodiments 293-298, wherein the level of the anti-inflammatory cytokine is increased by about 95% or more relative to a reference standard.

306. A composition for use in reducing the level of a hypersensitivity marker in a subject, wherein the composition for use comprises a combination of:
(i) a flavonoid; and
(ii) a xanthone.

307. The composition for use of embodiment 306, wherein following administration of the composition to the subject, the level of the hypersensitivity marker in the subject is reduced.

308. The composition for use of embodiment 307, wherein the hypersensitivity marker is selected from IL-13, IL-14, INF-g, and serum IgE.

309. The composition for use of any one of embodiments 306-308, wherein the hypersensitivity marker is IL-13.

310. The composition for use of any one of embodiments 306-308, wherein the hypersensitivity marker is IL-14.

311. The composition for use of any one of embodiments 306-308, wherein the hypersensitivity marker is INF-g.

312. The composition for use of any one of embodiments 306-308, wherein the hypersensitivity marker is serum IgE.

313. The composition for use of any one of embodiments 306-312, wherein the level of the hypersensitivity marker is reduced by about 10%, 25%, 50%, 75%, 90%, 95%, or more relative to a reference standard.

314. The composition for use of any one of embodiments 306-313, wherein the level of the hypersensitivity marker is reduced by about 10% or more relative to a reference standard.

315. The composition for use of any one of embodiments 306-313, wherein the level of the hypersensitivity marker is reduced by about 25% or more relative to a reference standard.

316. The composition for use of any one of embodiments 306-313, wherein the level of the hypersensitivity marker is reduced by about 50% or more relative to a reference standard.

317. The composition for use of any one of embodiments 306-313, wherein the level of the hypersensitivity marker is reduced by about 75% or more relative to a reference standard.

318. The composition for use of any one of embodiments 306-313, wherein the level of the hypersensitivity marker is reduced by about 90% or more relative to a reference standard.

319. The composition for use of any one of embodiments 306-313, wherein the level of the hypersensitivity marker is reduced by about 95% or more relative to a reference standard.

320. A composition for use in inducing expression of an anti-inflammatory macrophage in a subject, wherein the composition for use comprises a combination of:
(i) a flavonoid; and
(ii) a xanthone.

321. The composition for use of embodiment 320, wherein following administration of the composition to the subject, expression of the anti-inflammatory macrophage in the subject is induced.

322. The composition for use of any one of embodiments 320-321, wherein the anti-inflammatory macrophage is an M2 macrophage.

323. The composition for use of any one of embodiments 320-322, wherein the M2 macrophages secrete an anti-inflammatory cytokine.

324. The composition for use of embodiment 323, wherein the inflammatory cytokine comprises IL-10.

325. A composition for use in improving skin health in a subject, wherein the composition for use comprises a combination of:
(i) a flavonoid; and
(ii) a xanthone.

326. The composition for use of embodiment 325, wherein following administration of the composition to the subject, the skin health in the subject is improved.

327. A composition for use in reducing inflammation in a subject, wherein the composition for use comprises a combination of:
(i) a flavonoid; and
(ii) a xanthone.

328. The composition for use of embodiment 327, wherein following administration of the composition to the subject, the inflammation in the subject is reduced.

329. The composition for use of any one of embodiments 327-328, wherein the inflammation is dermal inflammation or epithelial cell inflammation.

330. The composition for use of any one of embodiments 327-329, wherein the inflammation is dermal inflammation.

331. The composition for use of any one of embodiments 327-329, wherein the inflammation is epithelial cell inflammation.

332. A composition for use in treating a dermatological condition in a subject, wherein the composition for use comprises a combination of:
(i) a flavonoid; and
(ii) a xanthone.

333. The composition for use of embodiment 332, wherein following administration of the composition to the subject, the dermatological condition in the subject is ameliorated.

334. The composition for use of any one of embodiments 332-333, wherein the dermatological condition is selected from atopic dermatitis, psoriasis, and dermal hypersensitivity.

335. The composition for use of any one of embodiments 332-334, wherein the dermatological condition is atopic dermatitis.

336. The composition for use of any one of embodiments 332-334, wherein the dermatological condition is psoriasis.

337. The composition for use of any one of embodiments 332-334, wherein the dermatological condition is dermal hypersensitivity.

338. A composition for use in treating topical inflammation in a subject, wherein the composition for use comprises a combination of:
(i) a flavonoid; and
(ii) a xanthone.

339. The composition for use of embodiment 338, wherein administration of the composition to the subject treats topical inflammation in the subject.

340. The composition for use of any one of embodiments 338-339, wherein the topical inflammation comprises hives or a rash.

341. The composition for use of any one of embodiments 338-340, wherein the topical inflammation comprises hives.

342. The composition for use of any one of embodiments 338-340, wherein the topical inflammation comprises a rash.

343. A composition for use in treating an inflammatory disease or disorder in a subject, wherein the composition for use comprises a combination of:
(i) a flavonoid; and
(ii) a xanthone.

344. The composition for use of embodiment 343, wherein following administration of the composition to the subject, the inflammatory disease or disorder in the subject is ameliorated.

345. The composition for use of any one of embodiments 343-344, wherein the inflammatory disease or disorder condition comprises rheumatoid arthritis.

346. A composition for use in reducing a symptom of inflammation or an inflammatory disease or disorder in a subject, wherein the composition for use comprises a combination of:
(i) a flavonoid; and
(ii) a xanthone.

347. The composition for use of embodiment 346, wherein following administration of the composition to the subject, the symptom of inflammation or an inflammatory disease or disorder in the subject is reduced.

348. The composition for use of any one of embodiments 346-347, wherein the symptom comprises swelling, irritation, redness, itching, hives, or sneezing.

349. The composition for use of any one of embodiments 346-348, wherein the symptom comprises swelling.

350. The composition for use of any one of embodiments 346-348, wherein the symptom comprises irritation.

351. The composition for use of any one of embodiments 346-348, wherein the symptom comprises redness.

352. The composition for use of any one of embodiments 346-348, wherein the symptom comprises itching.

353. The composition for use of any one of embodiments 346-348, wherein the symptom comprises hives.

354. The composition for use of any one of embodiments 346-348, wherein the symptom comprises sneezing.

355. The composition for use of any one of embodiments 276-354, wherein one of the flavonoid and the xanthone comprises an AHR agonist, an NRF2 agonist, or a PPARg agonist.

356. The composition for use of any one of embodiments 276-355, wherein one of the flavonoid and the xanthone comprises an AHR agonist.

357. The composition for use of any one of embodiments 276-355, wherein one of the flavonoid and the xanthone comprises an NRF2 agonist.

358. The composition for use of any one of embodiments 276-355, wherein one of the flavonoid and the xanthone comprises an PPARg agonist.

359. The composition for use of any one of embodiments 276-358, wherein each of the flavonoid and the xanthone independently comprises an AHR agonist, an NRF2 agonist, or a PPARg agonist.

360. The composition for use of any one of embodiments 276-359, wherein each of the flavonoid and the xanthone independently comprises an AHR agonist.

361. The composition for use of any one of embodiments 276-359, wherein each of the flavonoid and the xanthone independently comprises an NRF2 agonist.

362. The composition for use of any one of embodiments 276-359, wherein each of the flavonoid and the xanthone independently comprises an PPARg agonist.

363. The composition for use of any one of embodiments 276-362, wherein the xanthone has the structure of Formula (B):

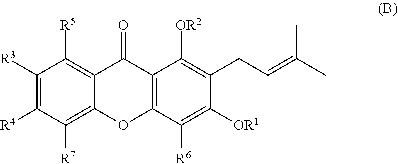

or a pharmaceutically acceptable salt thereof, wherein:
each of R¹ and R² is independently hydrogen or C₁-C₆ alkyl;
each of R³, R⁴, and R⁷ is independently hydrogen, —OR^{A}, C₁-C₆ alkyl, C₂-C₆ alkenyl, C₂-C₆ alkynyl, or C₂-C₁₀ alkenyl;
each of R⁵ and R⁶ is independently hydrogen, C₁-C₆ alkyl, C₂-C₆ alkenyl, C₂-C₆ alkynyl, —OR^{A} or C₂-C₁₀ alkenyl; and
R^{A} is hydrogen, C₁-C₆ alkyl, or cycloalkyl.

364. The composition for use of any one of embodiments 276-362, wherein the xanthone has the structure of Formula (B-i):

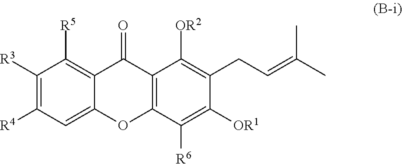

or a pharmaceutically acceptable salt thereof, wherein:
each of R¹ and R² is independently hydrogen or C₁-C₆ alkyl;
each of R³ and R⁴ is independently hydrogen or —OR^{A};
each of R⁵ and R⁶ is independently hydrogen, C₁-C₆ alkyl, C₂-C₆ alkenyl, C₂-C₆ alkynyl, or —OR^{A}; and
R^{A} is hydrogen, C₁-C₆ alkyl, or cycloalkyl.

365 The composition for use of embodiment 364, wherein R¹ is hydrogen.

366. The composition for use of any one of embodiments 364-365, wherein R² is hydrogen.

367 The composition for use of any one of embodiments 364-366, wherein R³ is —OR^{A}.

368. The composition for use of any one of embodiments 364-366, wherein R³ is —OH.

369. The composition for use of any one of embodiments 364-368, wherein R is —OR^{A}.

370. The composition for use of any one of embodiments 364-368, wherein R⁴ is —OH.

371. The composition for use of any one of embodiments 364-370, wherein R⁵ is C₂-C₆ alkenyl.

372, The composition for use of any one of embodiments 364-370, wherein R⁵ is —CH₂CH═C(CH₃)₂.

373. The composition for use of any one of embodiments 364-372, wherein R⁶ is hydrogen.

374. The composition for use of any one of embodiments 364-373, wherein each of R¹ and R² is hydrogen; each of R³ and R⁴ is —OR^{A}; R⁵ is C₂-C₆ alkenyl; and R⁶ is hydrogen.

375. The composition for use of any one of embodiments 276-362, wherein the xanthone has the structure of Formula (B-ii):

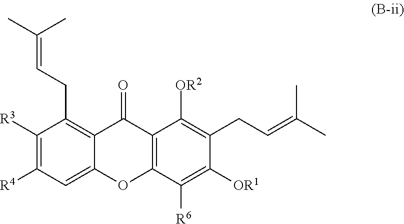

or a pharmaceutically acceptable salt thereof, wherein:
each of R¹ and R² is independently hydrogen or C₁-C₆ alkyl;
each of R³ and R⁴ is independently hydrogen or —OR^{A};
R⁶ is hydrogen, C₁-C₆ alkyl, C₂-C₆ alkenyl, C₁-C₆ alkynyl, or —OR^{A}; and
R^{A} is hydrogen, C₁-C₆ alkyl, or cycloalkyl.

376. The composition for use of embodiment 375, wherein each of R¹ and R² is hydrogen; each of R³ and R⁴ is —OR^{A}; and R⁶ is hydrogen.

377. The composition for use of any one of embodiments 276-362, wherein the xanthone is a compound of Formula (B-iii).

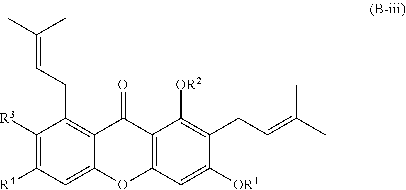

or a pharmaceutically acceptable salt thereof, wherein:
each of R¹ and R² is independently hydrogen or C₁-C₆ alkyl;
each of R³ and R⁴ is independently hydrogen or —OR^{A}; and
R^{A} is hydrogen, C₁-C₆ alkyl, or cycloalkyl.

378. The composition for use of embodiment 377, wherein each of R¹ and R² is hydrogen; and each of R³ and R⁴ is —OR^{A}.

379. The composition for use of embodiment 377, wherein each of R¹ and R² is hydrogen; R³ is —OCH₃, and R⁴ is —OH.

380. The composition for use of any one of embodiments 276-379, wherein the xanthone is selected from alpha-mangostin, beta-mangostin, gamma-mangostin, 8-deoxygartanin, garcinone E, gartanin, 1,7-dihydroxy-3-methoxy-2-prenylxanthone, 1,7-dihydroxy-3,6-dimethoxy-2,8-diprenylxanthone, morusignin B, dulxanthone B, 1,5-dihydroxy-3-methoxy-2-prenylxanthone, 1,4,6-trihydroxy-5-methoxy-7-prenylxanthone, nigrolineaxanthone E, 1-Hydroxy-3,5-dimethoxy-2,4-diprenylxanthone, gerontoxanthone I, 6-deoxy-gamma-mangostin, cowanin, dulcisxanthone B, cowaxanthone B, 1,3,7-trihydroxy-2,4-diisoprenylxanthone, cratoxyarborenone B, 1,3,5-trihydroxy-2-prenyl-9H-xanthene-9-one, 1,3,7-trihydroxy-2-prenylxanthone, doitunggarcinone C, cudratricusxanthone K, 1-hydroxy-3,5-dimethoxy-2-prenylxanthone, 1,8-dihydroxy-3,5-dimethoxy-2-prenylxanthone, or a pharmaceutically acceptable salt thereof.

381. The composition for use of any one of embodiments 276-380, wherein the xanthone is selected from alpha-mangostin, beta-mangostin, gamma-mangostin, 8-deoxygartanin, garcinone E, gartanin, or a pharmaceutically acceptable salt thereof.

382. The composition for use of any one of embodiments 276-381, wherein the xanthone is selected from alpha-mangostin, beta-mangostin, gamma-mangostin, and 8-deoxygartanin, or a pharmaceutically acceptable salt thereof.

383. The composition for use of any one of embodiments 276-382, wherein the xanthone is selected from alpha-mangostin, gamma-mangostin, or a pharmaceutically acceptable salt thereof.

384. The composition for use of any one of embodiments 276-382, wherein the xanthone is gamma-mangostin or a pharmaceutically acceptable salt thereof.

385. The composition for use of any one of embodiments 276-384, wherein the flavonoid has the structure of Formula (A):

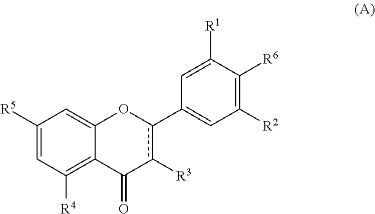

or a pharmaceutically acceptable salt thereof, wherein:
each of R¹, R², R³, R⁴, R⁵, and R⁶ is independently hydrogen or —OR^{A};
R^{A} is hydrogen, C₁-C₆ alkyl, or cycloalkyl, and
“ [symbol] ” is a single or double bond.

386. The composition for use of any one of embodiments 276-384, wherein the flavonoid has the structure of Formula (A-i):

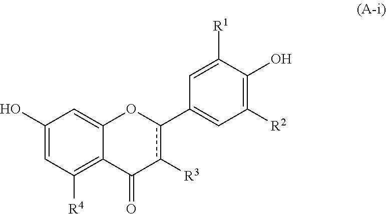

or a pharmaceutically acceptable salt thereof, wherein:
each of R¹ and R² is independently hydrogen or OR^{A};
R³ is hydrogen or OR^{A};
R⁴ is hydrogen or —OR^{A};
R^{A} is hydrogen, C₁-C₆ alkyl, or cycloalkyl; and
“ [symbol] ” is a single or double bond.

387. The composition for use of any one of embodiments 276-384, wherein the flavonoid has the structure of Formula (A-ii):

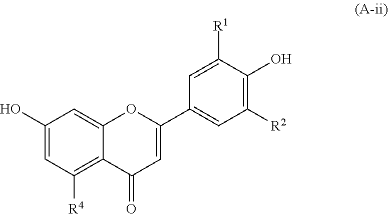

or a pharmaceutically acceptable salt thereof, wherein:
each of R¹ and R² is independently hydrogen or —OR^{A};
R⁴ is hydrogen or —OR^{A}; and
R^{A} is hydrogen, C₁-C₆ alkyl, or cycloalkyl.

388. The composition for use of embodiment 387, wherein R¹ is —OR^{A}; R² is hydrogen; and R⁴ is —OR^{A}.

389. The composition for use of embodiment 387, wherein R¹ is —OH; R² is hydrogen; and R⁴ is —OH.

390. The composition for use of any one of embodiments 276-384, wherein the flavonoid has the structure of Formula (A-iii):

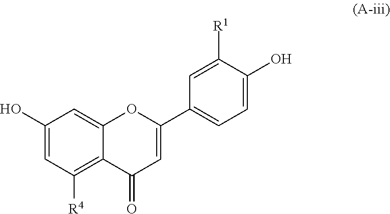

or a pharmaceutically acceptable salt thereof, wherein:
each of R¹ is hydrogen or —OR^{A};
R⁴ is hydrogen or —OR^{A}; and
R^{A} is hydrogen, C₁-C₆ alkyl, or cycloalkyl.

391. The composition for use of embodiment 390, wherein R¹ is —OR^{A} and R⁴ is —OR A.

392. The composition for use of embodiment 390, wherein R¹ is —OH and R⁴ is —OH.

393. The composition for use of any one of embodiments 276-392, wherein the flavonoid is selected from quercetin, luteolin, myricetin, taxifolin, fisetin, kaempferol, apigenin, tricetin, isohamnetin, eriodictyol, dihydromyricetin, chryseriol, 7,3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin, or a pharmaceutically acceptable salt thereof.

394. The composition for use of any one of embodiments 276-393, wherein the flavonoid is selected from quercetin, luteolin, myricetin, taxifolin, fisetin, kaempferol, or a pharmaceutically acceptable salt thereof.

395. The composition for use of any one of embodiments 276-394, wherein the flavonoid is selected from luteolin, myricetin, taxifolin, fisetin, kaempferol, or a pharmaceutically acceptable salt thereof.

396. The composition for use of any one of embodiments 276-395, wherein the flavonoid is quercetin, luteolin, or a pharmaceutically acceptable salt thereof.

397. The composition for use of any one of embodiments 276-396, wherein the flavonoid is luteolin or a pharmaceutically acceptable salt thereof.

398. The composition for use of any one of embodiments 276-397, wherein the efficacy of the combination is at least X₁-fold greater than the efficacy of the flavonoid alone at the molar amount used in the combination, wherein X₁ is 1, 1.25, 1.5, 1.75, 2, 2.5, or greater.

399. The composition for use of any one of embodiments 276-398, wherein the efficacy of the combination is at least X₁-fold greater than the efficacy of the flavonoid alone at the molar amount used in the combination, wherein X₁ is 1 or greater.

400. The composition for use of any one of embodiments 276-398, wherein the efficacy of the combination is at least X₁-fold greater than the efficacy of the flavonoid alone at the molar amount used in the combination, wherein X₁ is 1.25 or greater.

401. The composition for use of any one of embodiments 276-398, wherein the efficacy of the combination is at least X₁-fold greater than the efficacy of the flavonoid alone at the molar amount used in the combination, wherein X₁ is 1.5 or greater.

402. The composition for use of any one of embodiments 276-398, wherein the efficacy of the combination is at least X₁-fold greater than the efficacy of the flavonoid alone at the molar amount used in the combination, wherein X₁ is 1.75 or greater.

403. The composition for use of any one of embodiments 276-398, wherein the efficacy of the combination is at least X₁-fold greater than the efficacy of the flavonoid alone at the molar amount used in the combination, wherein X₁ is 2 or greater.

404. The composition for use of any one of embodiments 276-398, wherein the efficacy of the combination is at least X₁-fold greater than the efficacy of the flavonoid alone at the molar amount used in the combination, wherein X₁ is 2.5 or greater.

405. The composition for use of any one of embodiments 276-404, wherein the efficacy of the combination is at least X₁-fold greater than the efficacy of the xanthone alone at the molar amount used in the combination, wherein X₁ is 1, 1.25, 1.5, 1.75, 2, 2.5, or greater.

406. The composition for use of any one of embodiments 276-405, wherein the efficacy of the combination is at least X₁-fold greater than the efficacy of the xanthone alone at the molar amount used in the combination, wherein X₁ is 1 or greater.

407. The composition for use of any one of embodiments 276-405, wherein the efficacy of the combination is at least X₁-fold greater than the efficacy of the xanthone alone at the molar amount used in the combination, wherein X₁ is 1.25 or greater.

408. The composition for use of any one of embodiments 276-405, wherein the efficacy of the combination is at least X₁-fold greater than the efficacy of the xanthone alone at the molar amount used in the combination, wherein X₁ is 1.5 or greater.

409. The composition for use of any one of embodiments 276-405, wherein the efficacy of the combination is at least X₁-fold greater than the efficacy of the xanthone alone at the molar amount used in the combination, wherein X₁ is 1.75 or greater.

410. The composition for use of any one of embodiments 276-405, wherein the efficacy of the combination is at least X₁-fold greater than the efficacy of the xanthone alone at the molar amount used in the combination, wherein X₁ is 2 or greater.

411. The composition for use of any one of embodiments 276-405, wherein the efficacy of the combination is at least X₁-fold greater than the efficacy of the xanthone alone at the molar amount used in the combination, wherein X₁ is 2.5 or greater.

412. The composition for use of any one of embodiments 276-411, wherein the xanthone is gamma-mangostin and the flavonoid is luteolin.

413. The composition for use of any one of embodiments 276-412, wherein the xanthone is prepared synthetically.

414. The composition for use of any one of embodiments 276-413, wherein the flavonoid is prepared synthetically.

415. The composition for use of any one of embodiments 276-414, wherein the xanthone and/or the flavonoid is extracted from a natural source (e.g., a plant).

416. The composition for use of any one of embodiments 276-415, wherein the flavonoid is a compound selected from Table 1 or a pharmaceutically acceptable salt thereof.

417. The composition for use of embodiment 416, wherein the flavonoid is substantially pure.

418. The composition for use of any one of embodiments 276-417, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of another compound.

419. The composition for use of embodiment 418, wherein the pharmaceutical composition comprises less than about 50% of another compound.

420. The composition for use of embodiment 418, wherein the pharmaceutical composition comprises less than about 60% of another compound.

421. The composition for use of embodiment 418, wherein the pharmaceutical composition comprises less than about 65% of another compound.

422. The composition for use of embodiment 418, wherein the pharmaceutical composition comprises less than about 70% of another compound.

423. The composition for use of embodiment 418, wherein the pharmaceutical composition comprises less than about 75% of another compound.

424. The composition for use of embodiment 418, wherein the pharmaceutical composition comprises less than about 80% of another compound.

425. The composition for use of embodiment 418, wherein the pharmaceutical composition comprises less than about 85% of another compound.

426. The composition for use of embodiment 418, wherein the pharmaceutical composition comprises less than about 90% of another compound.

427. The composition for use of embodiment 418, wherein the pharmaceutical composition comprises less than about 95% of another compound.

428. The composition for use of embodiment 418, wherein the pharmaceutical composition comprises less than about 99% of another compound.

429. The composition for use of embodiment 418, wherein the pharmaceutical composition comprises less than about 99.9% of another compound.

430. The composition for use of any one of embodiments 276-429, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of another flavonoid, such as a flavonoid listed in Table 1 or a pharmaceutically acceptable salt thereof.

431. The composition for use of embodiment 430, wherein the pharmaceutical composition comprises less than about 50% of another flavonoid.

432. The composition for use of embodiment 430, wherein the pharmaceutical composition comprises less than about 60% of another flavonoid.

433. The composition for use of embodiment 430, wherein the pharmaceutical composition comprises less than about 65% of another flavonoid.

434. The composition for use of embodiment 430, wherein the pharmaceutical composition comprises less than about 70% of another flavonoid.

435. The composition for use of embodiment 430, wherein the pharmaceutical composition comprises less than about 75% of another flavonoid.

436. The composition for use of embodiment 430, wherein the pharmaceutical composition comprises less than about 80% of another flavonoid.

437. The composition for use of embodiment 430, wherein the pharmaceutical composition comprises less than about 85% of another flavonoid.

438. The composition for use of embodiment 430, wherein the pharmaceutical composition comprises less than about 90% of another flavonoid.

439. The composition for use of embodiment 430, wherein the pharmaceutical composition comprises less than about 95% of another flavonoid.

440. The composition for use of embodiment 430, wherein the pharmaceutical composition comprises less than about 99% of another flavonoid.

441. The composition for use of embodiment 430, wherein the pharmaceutical composition comprises less than about 99.9% of another flavonoid.

442. The composition for use of any one of embodiments 276-441, wherein the flavonoid is provided as a pharmaceutical composition and the pharmaceutical composition comprises less than about 50%, 55%, 60%, 65%, 70%, 75%, 80%, 85%, 90%, 95%, 99%, or 99.9% of a plant-derived substance.

443. The composition for use of embodiment 442, wherein the pharmaceutical composition comprises less than about 50/a of a plant-derived substance.

444. The composition for use of embodiment 442, wherein the pharmaceutical composition comprises less than about 55% of a plant-derived substance.

445. The composition for use of embodiment 442, wherein the pharmaceutical composition comprises less than about 60% of a plant-derived substance.

446. The composition for use of embodiment 442, wherein the pharmaceutical composition comprises less than about 65% of a plant-derived substance.

447. The composition for use of embodiment 442, wherein the pharmaceutical composition comprises less than about 70% of a plant-derived substance.

448. The composition for use of embodiment 442, wherein the pharmaceutical composition comprises less than about 75% of a plant-derived substance.

449. The composition for use of embodiment 442, wherein the pharmaceutical composition comprises less than about 80% of a plant-derived substance.

450. The composition for use of embodiment 442, wherein the pharmaceutical composition comprises less than about 85% of a plant-derived substance.

451. The composition for use of embodiment 442, wherein the pharmaceutical composition comprises less than about 90% of a plant-derived substance.

452. The composition for use of embodiment 442, wherein the pharmaceutical composition comprises less than about 95% of a plant-derived substance.

453. The composition for use of embodiment 442, wherein the pharmaceutical composition comprises less than about 99% of a plant-derived substance.

454. The composition for use of embodiment 442, wherein the pharmaceutical composition comprises less than about 99.9% of a plant-derived substance.

455. The composition for use of any one of embodiments 276-454, wherein the flavonoid is provided in the combination in the absence of quercetin, luteolin, myricetin, taxifolin, fisetin, kaempferol, apigenin, tricetin, isohamnetin, eriodictyol, dihydromyricetin, chryseriol, 7,3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin or a pharmaceutically acceptable salt thereof.

456. The composition for use of any one of embodiments 276-455, wherein the flavonoid is luteolin or a pharmaceutically acceptable salt thereof.

457. The composition for use of embodiment 456, wherein the luteolin or a pharmaceutically acceptable salt thereof is provided in the combination in the absence of quercetin, luteolin, myricetin, taxifolin, fisetin, kaempferol, apigenin, tricetin, isohamnetin, eriodictyol, dihydromyricetin, chryseriol, 7,3′,4′-trihydroxyflavone, 4′7-dihydroxyflavone, hesperitin, galangin, chrysin, 7-O-methylluteolin, genkwanin, acacetin, retusin, 7-hydroxyflavone, apigenin 7,4′-dimethyl ether, pratol, ombuin, quercetin 3,3′-dimethyl ether, tectochrysin, tricin, or a pharmaceutically acceptable salt thereof.

458. The composition for use of any one of embodiments 456-457, wherein the luteolin or a pharmaceutically acceptable salt thereof is provided as a pharmaceutical composition, and the pharmaceutical composition comprises less than about 90%, 95%, 99%, or 99.9% of another compound in listed in Table 1 or a pharmaceutically acceptable salt thereof.

459. The composition for use of embodiment 458, wherein the pharmaceutical composition comprises less than about 90% of another compound listed in Table 1 or a pharmaceutically acceptable salt thereof.

460. The composition for use of embodiment 458, wherein the pharmaceutical composition comprises less than about 95% of another compound listed in Table 1 or a pharmaceutically acceptable salt thereof.

461. The composition for use of embodiment 458, wherein the pharmaceutical composition comprises less than about 99% of another compound listed in Table 1 or a pharmaceutically acceptable salt thereof.

462. The composition for use of embodiment 458, wherein the pharmaceutical composition comprises less than about 99.9% of another compound listed in Table 1 or a pharmaceutically acceptable salt thereof.

463. The composition for use of any one of embodiments 276-462, wherein the xanthone is a compound selected from Table 1 or a pharmaceutically acceptable salt thereof.

464. The composition for use of embodiment 463, wherein the xanthone is substantially pure.

465. The composition for use of any one of embodiments 463-464, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of another compound.

466. The composition for use of any one of embodiment 465, wherein the pharmaceutical composition comprises less than about 50% of another compound.

467. The composition for use of any one of embodiment 465, wherein the pharmaceutical composition comprises less than about 55% of another compound.

468. The composition for use of any one of embodiment 465, wherein the pharmaceutical composition comprises less than about 60% of another compound.

469. The composition for use of any one of embodiment 465, wherein the pharmaceutical composition comprises less than about 65% of another compound.

470. The composition for use of any one of embodiment 465, wherein the pharmaceutical composition comprises less than about 70% of another compound.

471. The composition for use of any one of embodiment 465, wherein the pharmaceutical composition comprises less than about 75% of another compound.

472. The composition for use of any one of embodiment 465, wherein the pharmaceutical composition comprises less than about 80% of another compound.

473. The composition for use of any one of embodiment 465, wherein the pharmaceutical composition comprises less than about 85% of another compound.

474. The composition for use of any one of embodiment 465, wherein the pharmaceutical composition comprises less than about 90% of another compound.

475. The composition for use of any one of embodiment 465, wherein the pharmaceutical composition comprises less than about 95% of another compound.

476. The composition for use of any one of embodiment 465, wherein the pharmaceutical composition comprises less than about 99% of another compound.

477. The composition for use of any one of embodiment 465, wherein the pharmaceutical composition comprises less than about 99.9% of another compound.

478. The composition for use of any one of embodiments 276-477, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of another xanthone, such as a xanthone listed in Table 2 or a pharmaceutically acceptable salt thereof.

479. The composition for use of embodiment 478, wherein the pharmaceutical composition comprises less than about 50% of another xanthone.

480. The composition for use of embodiment 478, wherein the pharmaceutical composition comprises less than about 55% of another xanthone.

481. The composition for use of embodiment 478, wherein the pharmaceutical composition comprises less than about 60% of another xanthone.

482. The composition for use of embodiment 478, wherein the pharmaceutical composition comprises less than about 65% of another xanthone.

483. The composition for use of embodiment 478, wherein the pharmaceutical composition comprises less than about 70% of another xanthone.

484. The composition for use of embodiment 478, wherein the pharmaceutical composition comprises less than about 75% of another xanthone.

485. The composition for use of embodiment 478, wherein the pharmaceutical composition comprises less than about 80% of another xanthone.

486. The composition for use of embodiment 478, wherein the pharmaceutical composition comprises less than about 85% of another xanthone.

487. The composition for use of embodiment 478, wherein the pharmaceutical composition comprises less than about 90% of another xanthone.

488. The composition for use of embodiment 478, wherein the pharmaceutical composition comprises less than about 95% of another xanthone.

489. The composition for use of embodiment 478, wherein the pharmaceutical composition comprises less than about 99% of another xanthone.

490. The composition for use of embodiment 478, wherein the pharmaceutical composition comprises less than about 99.9% of another xanthone.

491. The composition for use of any one of embodiments 276-490, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of a plant-derived substance.

492. The composition for use of embodiment 491, wherein the pharmaceutical composition comprises less than about 50% of a plant-derived substance.

493. The composition for use of embodiment 491, wherein the pharmaceutical composition comprises less than about 55% of a plant-derived substance.

494. The composition for use of embodiment 491, wherein the pharmaceutical composition comprises less than about 60% of a plant-derived substance.

495. The composition for use of embodiment 491, wherein the pharmaceutical composition comprises less than about 65% of a plant-derived substance.

496. The composition for use of embodiment 491, wherein the pharmaceutical composition comprises less than about 70% of a plant-derived substance.

497. The composition for use of embodiment 491, wherein the pharmaceutical composition comprises less than about 75% of a plant-derived substance.

498. The composition for use of embodiment 491, wherein the pharmaceutical composition comprises less than about 80% of a plant-derived substance.

499. The composition for use of embodiment 491, wherein the pharmaceutical composition comprises less than about 85% of a plant-derived substance.

500. The composition for use of embodiment 491, wherein the pharmaceutical composition comprises less than about 90% of a plant-derived substance.

501. The composition for use of embodiment 491, wherein the pharmaceutical composition comprises less than about 95% of a plant-derived substance.

502. The composition for use of embodiment 491, wherein the pharmaceutical composition comprises less than about 99% of a plant-derived substance.

503. The composition for use of embodiment 491, wherein the pharmaceutical composition comprises less than about 99.9% of a plant-derived substance.

504. The composition for use of any one of embodiments 276-503, wherein the flavonoid is provided in the combination in the absence of hyperforin, adhyperforin, or a pharmaceutically acceptable salt thereof.

505. The composition for use of any one of embodiments 276-504, wherein the xanthone is gamma-mangostin or a pharmaceutically acceptable salt thereof.

506. The composition for use of embodiment 505, wherein the gamma-mangostin or a pharmaceutically acceptable salt thereof is provided in the combination in the absence of alpha-mangostin, beta-mangostin, 8-deoxygartanin, garcinone E, gartanin, 1,7-dihydroxy-3-methoxy-2-prenylxanthone, 1,7-dihydroxy-3,6-dimethoxy-2,8-diprenylxanthone, morusignin B, dulxanthone B, 1,5-dihydroxy-3-methoxy-2-prenylxanthone, 1,4,6-trihydroxy-5-methoxy-7-prenylxanthone, nigrolineaxanthone E, 1-Hydroxy-3,5-dimethoxy-2,4-diprenylxanthone, gerontoxanthone I, 6-deoxy-gamma-mangostin, cowanin, dulcisxanthone B, cowaxanthone B, 1,3,7-trihydroxy-2,4-diisoprenylxanthone, cratoxyarborenone B, 1,3,5-trihydroxy-2-prenyl-9H-xanthene-9-one, 1,3,7-trihydroxy-2-prenylxanthone, doitunggarcinone C, cudratricusxanthone K, 1-hydroxy-3,5-dimethoxy-2-prenylxanthone, 1,8-dihydroxy-3,5-dimethoxy-2-prenylxanthone, or a pharmaceutically acceptable salt thereof.

507. The composition for use of any one of embodiments 276-506, wherein the luteolin or a pharmaceutically acceptable salt thereof is provided as a pharmaceutical composition, and the pharmaceutical composition comprises less than about 90%, 95%, 99%, or 99.9% of another compound listed in Table 2 or a pharmaceutically acceptable salt thereof.

508. The composition for use of embodiment 507, wherein the pharmaceutical composition comprises less than about 90% of another compound listed in Table 2 or a pharmaceutically acceptable salt thereof.

509. The composition for use of embodiment 507, wherein the pharmaceutical composition comprises less than about 95% of another compound listed in Table 2 or a pharmaceutically acceptable salt thereof.

510. The composition for use of embodiment 507, wherein the pharmaceutical composition comprises less than about 99% of another compound listed in Table 2 or a pharmaceutically acceptable salt thereof.

511. The composition for use of embodiment 507, wherein the pharmaceutical composition comprises less than about 99.9% of another compound listed in Table 2 or a pharmaceutically acceptable salt thereof.

512. The composition for use of any one of embodiments 276-511, wherein each of gamma-mangostin and luteolin is independently formulated as a pharmaceutical composition.

513. The composition for use of any one of embodiments 276-511, wherein the gamma-mangostin and luteolin are formulated together as a pharmaceutical composition.

514. The composition for use of any one of embodiments 276-513, wherein each of the gamma-mangostin and luteolin is provided concomitantly to the subject.

515. The composition for use of embodiment 514, wherein each of the gamma-mangostin and luteolin is provided sequentially to the subject.

516. The composition for use of embodiment 515, wherein the gamma-mangostin is provided to the subject prior to the luteolin.

517. The composition for use of embodiment 516, wherein the luteolin is provided to the subject prior to the gamma-mangostin.

518. The composition for use of any one of embodiments 276-517, wherein the xanthone and the flavonoid are formulated for topical, systemic, or oral administration.

519. The composition for use of embodiment 518, wherein the xanthone and the flavonoid are formulated for topical administration.

520. The composition for use of embodiment 518, wherein the xanthone and the flavonoid are formulated for systemic administration.

521. The composition for use of embodiment 518, wherein the xanthone and the flavonoid are formulated for oral administration.

522. The composition for use of any one of embodiments 276-521, wherein the flavonoid is administered at a dosage to provide a concentration of between 0.1 μM and 500 μM in the subject or a cell.

523. The composition for use of any one of embodiments 276-522, wherein the flavonoid is administered at a dosage to provide a concentration of between 1 μM and 25 μM in the subject or a cell.

524. The composition for use of any one of embodiments 276-522, wherein the xanthone is administered at a dosage to provide a concentration of between 0.01 μM and 25 μM in the subject or a cell.

525. The composition for use of any one of embodiments 276-522, wherein the xanthone is administered at a dosage to provide a concentration of between 0.1 μM and 5 μM in the subject or a cell.

526. The composition for use of any one of embodiments 276-525, wherein the ratio of the amount of flavonoid to the xanthone in the combination is between 200:1 to 1:1.

527. The composition for use of embodiment 526, wherein the ratio of the amount of flavonoid to the xanthone in the combination is between 50:1 to 1:1.

528. The composition for use of any one of embodiments 526-527, wherein the ratio of the amount of flavonoid to the xanthone in the combination is between 50:1 to 2:1.

529. The composition for use of any one of embodiments 526-527, wherein the ratio of the amount of flavonoid to the xanthone in the combination is between 25:1 to 2:1.

530. The composition for use of any one of embodiments 276-529, wherein:
(i) the flavonoid is luteolin or a pharmaceutically acceptable salt thereof;
(ii) the xanthone is gamma-mangostin or a pharmaceutically acceptable salt thereof; and
(iii) the molar amount of the flavonoid in the combination is between 20-fold and 5-fold greater than the molar amount of the xanthone.

531. The composition for use of any one of embodiments 276-530, further comprising administration of an additional agent.

532. The composition for use of any one of embodiments 276-531, wherein the subject is a human.

533. The composition for use of any one of embodiments 276-532, wherein the inflammation is present within an inflamed organ, tissue, or cell in a subject.

534. The composition for use of embodiment 533, wherein the inflammation is present within an inflamed organ in the subject.

535. The composition for use of embodiment 534, wherein the inflamed organ is selected from the group consisting of the skin, brain, spinal cord, eye, lung, heart, pancreas, large intestine, small intestine, stomach, liver, gall bladder, kidney, or spleen.

536. The composition for use of embodiment 535, wherein the inflamed organ is the skin.

537. The composition for use of embodiment 535, wherein the inflamed organ is the brain.

538. The composition for use of embodiment 535, wherein the inflamed organ is the spinal cord.

539. The composition for use of embodiment 535, wherein the inflamed organ is the eye.

540. The composition for use of embodiment 535, wherein the inflamed organ is the lung.

541. The composition for use of embodiment 535, wherein the inflamed organ is the heart.

542. The composition for use of embodiment 535, wherein the inflamed organ is the pancreas.

543. The composition for use of embodiment 535, wherein the inflamed organ is the large intestine.

544. The composition for use of embodiment 535, wherein the inflamed organ is the small intestine.

545. The composition for use of embodiment 535, wherein the inflamed organ is the stomach.

546. The composition for use of embodiment 535, wherein the inflamed organ is the liver.

547. The composition for use of embodiment 535, wherein the inflamed organ is the gall bladder.

548. The composition for use of embodiment 535, wherein the inflamed organ is the kidney.

549. The composition for use of embodiment 535, wherein the inflamed organ is the spleen.

550. The composition for use of embodiment 535, wherein the inflammation is present within an inflamed tissue in the subject.

551. The composition for use of embodiment 550, wherein the inflamed tissue is selected from the group consisting of lung tissue, tracheal tissue, intestinal tissue, skin tissue, pancreatic tissue, vascular tissue, mucosal tissue, kidney tissue, brain tissue, nervous tissue, or cardiac tissue.

552. The composition for use of embodiment 551, wherein the inflamed tissue is lung tissue.

553. The composition for use of embodiment 551, wherein the inflamed tissue is tracheal tissue.

554. The composition for use of embodiment 551, wherein the inflamed tissue is intestinal tissue.

555. The composition for use of embodiment 551, wherein the inflamed tissue is skin tissue.

556. The composition for use of embodiment 551, wherein the inflamed tissue is pancreatic tissue.

557. The composition for use of embodiment 551, wherein the inflamed tissue is vascular tissue.

558. The composition for use of embodiment 551, wherein the inflamed tissue is mucosal tissue.

559. The composition for use of embodiment 551, wherein the inflamed tissue is kidney tissue.

560. The composition for use of embodiment 551, wherein the inflamed tissue is brain tissue.

561. The composition for use of embodiment 551, wherein the inflamed tissue is nervous tissue.

562. The composition for use of embodiment 551, wherein the inflamed tissue is cardiac tissue.

### EXAMPLES

The following examples are provided to further illustrate some embodiments of the present disclosure but are not intended to limit the scope of the disclosure. It will be understood by their exemplary nature that other procedures, methodologies, or techniques known to those skilled in the art may alternatively be used.

### Example 1: In Vitro Assay to Determine Anti-Inflammatory Properties of Exemplary Compounds—THP1 Cytokine Secretion Assay

AhR, NRF2, or PPARG agonism are known to lead to downstream inhibition of the secretion of various inflammatory cytokines (e.g., TNFa, IL6). Given that AhR, NRF2, or PPARG agonism were observed, the purpose of this example was to demonstrate the ability of luteolin and gamma-mangostin, alone or in combination, to impact various downstream, functional inflammatory processes.

Human THP-1 cells were plated and differentiated into macrophages through treatment with phorbol 12-myristate 13-acetate and beta-mercaptoethanol for 72 hours. Differentiated THP-1 macrophages were then stimulated with IFN-gamma (20 ng/mL) and LPS (50 ng/mL) to induce an inflammatory response and co-treated with gamma-mangostin and/or luteolin at 0, 0.2, 0.5, 2, 5, 10 uM. Various secreted cytokines were measured from the media using the Ella Automated Immunoassay System (ProteinSimple).

Luteolin alone (0-10 uM) led to inhibition of TNFa secretion, with the greatest TNFa inhibition to 45.6% seen at 0.5 uM. Gamma-mangostin alone (0-10 uM) also led to inhibition of TNFa secretion, with the greatest TNFa inhibition to 45% seen at 10 uM. Luteolin and Gamma-mangostin synergized when screened in combination at certain concentrations to lead to even greater TNFa inhibition than the max impact of each compound alone. As an example, Luteolin (5 uM) in combination with Gamma-mangostin (0.2 uM) led to TNFa inhibition to 20%, and Gamma-mangostin (5 uM) in combination with Luteolin (0.2-10 uM) led to TNFa inhibition up to 25.2%.

Luteolin alone (0-10 uM) led to inhibition of IL6 secretion, with the greatest IL6 inhibition to 53.7% seen at 0.5 uM. Gamma-mangostin alone (0-10 uM) also led to inhibition of IL6 secretion, with the greatest IL6 inhibition to 53% seen at 10 uM. Luteolin and Gamma-mangostin synergized when screened in combination at certain concentrations to lead to even greater IL6 inhibition than the max impact of each compound alone. As an example, Luteolin (0.2 uM) in combination with Gamma-mangostin (0.2-5 uM) led to IL6 inhibition up to 24.2%.

### Example 2: In Vitro Assay to Determine Anti-Inflammatory Properties of Exemplary Compounds—Primary T Cell Cytokine Secretion Assay

CD4+CD25− T-responder cells T-cells were isolated from human PBMC (STEMCELL Technologies, Cat #70025) using the CD4+CD127lowCD25+ regulatory T-cell isolation kit (Cat #18063, STEMCELL Technologies). For CD4+ T-responder cell cytokine release, cells were seeded at 25000/well in a V-bottom 96-well plate and exposed to IL-2 (100 U/mL) to trigger cytokine secretion. Luteolin+Gamma-mangostin (5 uM+5 uM) ability to prevent cytokine secretion was evaluated. A cytokine panel analysis was performed using Ella Automated Immunoassay System (ProteinSimple).

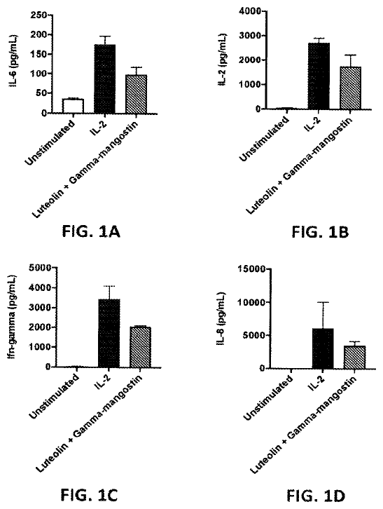

IL-6 secretion increased from 36 pg/mL in the Unstimulated group to 176 pg/mL after exposure to IL-2 (positive control). The combo Luteolin+Gamma-Mangostin partially prevented TL-6 secretion by T-cells (99 pg/mL compared to 176 ng/mL). Luteolin+Gamma-mangostin also prevented further IL-2 secretion induced by IL-2 (1750 versus 2715 ng/mL), as well as Ifn-gamma secretion (2052 versus 3462 ng/mL) and IL-8 secretion (3485 versus 6053 ng/mL) (FIGS. 1A-1D).

### Example 3: Oxazolone-Induced Dermatitis Mouse Model: Experimental Design and Results

The purpose of this example was to demonstrate the combinatorial, anti-inflammatory effects of a combination of Luteolin or Gamma-mangostin applied topically on oxazolone-induced ear swelling in mice, a model of allergic contact dermatitis. Male BALB/c mice weighing 22±2 g are used and randomly divided into groups of 5 each. Sham treatment group receive no oxazolone sensitization and challenge, but PBS, plus treated with vehicle (Acetone:ethanol 1:1). All other groups' animals were sensitized by applying oxazolone (100 μL, 1.5% in acetone) onto their preshaved abdominal surface on Day 0, and challenged by oxazolone (1%, 20 μL/ear) seven (7) days later. 20 μL of either Vehicle, Dexamethasone (5 mg/mL; anti-inflammatory positive control), or a combination of Luteolin (1.6-9.6 mg/mL)+Gamma-mangostin (1.2-7.2 mg/mL) were applied topically to the anterior and posterior surfaces of the right ear 30 minutes before and 15 minutes after oxazolone challenge. Left ear was unstimulated and treated as a control.

The right and left ear thickness of each mouse were measured at 24 hours after oxazolone challenge as an index of inflammation. The right ear was harvested and snap frozen for cytokine gene expression analysis. Each compound was assessed by calculating the percentage of cytokine gene expression compared to the Vehicle control, with a lower percentage indicating greater anti-inflammatory action of the compound.

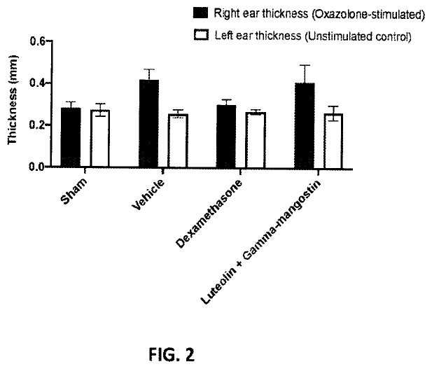

Vehicle-treated ears exhibited oxazolone-stimulated increases in ear thickness to 0.42 mm. Dexamethasone-treated ears demonstrated a 29% reduction in thickness compared to the Vehicle-treated ears. Luteolin+Gamma-mangostin-treated ears did not demonstrate any notable change in thickness compared to the Vehicle-treated ears (FIG. 2).

Vehicle-treated mice exhibited oxazolone-induced increases in expression of Il1b, Il4, Il6, Il11, Il13, and Tnfa compared to the Sham group. Topical treatment with Dexamethasone effectively reduced Il1b, Il4, Il6, Il11, Il13, and Tnfa gene expression down to 5%, 24%, 33%, 55%, 20%, and 14% of the Vehicle control, respectively. Dexamethasone increased Ifng gene expression to 445% of the Vehicle control. Topical treatment with Gamma-mangostin+Luteolin reduced Il1b, Il4, Il6, Il11, Il13, Tnfa, and Ifng down to 33%, 18%, 37%, 90%, 38%, 42%, and 49% of the Vehicle control, respectively (Table 3).

TABLE 3
Percentage of cytokine gene expression in ears of mice stimulated
with oxazolone and topically treated with Dexamethasone or
Luteolin + Gamma-mangostin compared to Vehicle control.

| Cytokine | Dexamethasone | Luteolin + Gamma-mangostin |
|---|---|---|
| gene | (% of Vehicle control) | (% of Vehicle control) |
| Il1b | 5% | 33% |
| Il4 | 24% | 18% |
| Il6 | 33% | 37% |
| Il11 | 55% | 90% |
| Il13 | 20% | 38% |
| Tnfa | 14% | 42% |
| Ifng | 445% | 49% |

### Example 4: LPS-Induced Systemic Inflammation Mouse Model: Experimental Design and Results

C57Bl/6 male mice, 6-8 weeks old, weighing 22±2 g were randomly divided into groups of 5-6 each. Sham animals were injected intra-peritoneally with PBS only. All other groups were intraperitoneally injected with either Vehicle control, Dexamethasone (5 mg/kg), or a combination of Luteolin+Gamma-mangostin (4+4.5 mg/kg) at 1 hour prior to injection with LPS (1 mg/kg), an agent known to trigger systemic inflammation. After LPS injection, blood was collected at different time points: 30 min, 2 h, 4 h and 24 h and the processed serum or plasma were used for further cytokine analysis through LUMINEX or Ella Automated Immunoassay System (Protein Simple).

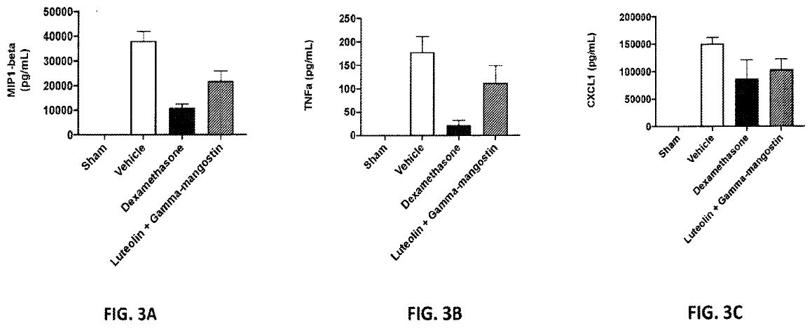

Dexamethasone reduced MIP1-beta, CXCL1, and TNFa levels down to 29%, 57%, and 12% of the Vehicle in LPS-injected mice, respectively. Luteolin+Gamma-mangostin reduced MIP1-beta, CXCL1, and TNFa levels down to 57%, 69%, and 63% of the Vehicle in LPS-injected mice, respectively (FIGS. 3A-3C).

### INCORPORATION BY REFERENCE AND EQUIVALENTS

The entire contents of all patents, published applications, and references cited herein are incorporated by reference in their entirety. While example embodiments have been particularly shown and described, it will be understood by those skilled in the art that various changes in form and details may be made therein without departing from the scope of the embodiments encompassed by the appended claims.

1. A composition for use in reducing inflammation in a mammal comprising a combination of:
(i) a flavonoid; and
(ii) a xanthone.

2. The composition for use of claim 1, wherein the inflammation is dermal inflammation or epithelial cell inflammation.

3. A composition for use in treating a dermatological condition in a mammal comprising a combination of:
(i) a flavonoid; and
(ii) a xanthone.

4. The composition for use of claim 3, wherein the dermatological condition is selected from atopic dermatitis, psoriasis, and dermal hypersensitivity.

5. The composition for use of any one of the preceding claims, wherein one of the flavonoid and the xanthone comprises an AHR agonist, an NRF2 agonist, or a PPARg agonist.

6. The composition for use of any one of the preceding claims, wherein each of the flavonoid and the xanthone independently comprises an AHR agonist, an NRF2 agonist, or a PPARg agonist.

7. The composition for use of any one of the preceding claims, wherein the xanthone has the structure of Formula (B-i): 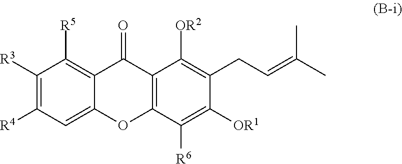
or a pharmaceutically acceptable salt thereof, wherein:
each of R¹ and R² is independently hydrogen or C₁-C₆ alkyl;
each of R³ and R⁴ is independently hydrogen or —OR^{A};
each of R⁵ and R⁶ is independently hydrogen, C₁-C₆ alkyl, C₂-C₆ alkenyl, C₂-C₆ alkynyl, or —OR^{A}; and
R^{A} is hydrogen, C₁-C₆ alkyl, or cycloalkyl.

8. The composition for use of claim 7, wherein the compound of Formula (B-i) is selected from a compound of Table 2.

9. The composition for use of any one of claims 7-8, wherein the compound of Formula (B-i) is selected from alpha-mangostin, beta-mangostin, gamma-mangostin, and 8-deoxygartanin, or a pharmaceutically acceptable salt thereof.

10. The composition for use of any one of the preceding claims, wherein the xanthone is selected from alpha-mangostin, gamma-mangostin, or a pharmaceutically acceptable salt thereof.

11. The composition for use of any one of the preceding claims, wherein the xanthone is gamma-mangostin or a pharmaceutically acceptable salt thereof.

12. The composition for use of any one of the preceding claims, wherein the flavonoid has the structure of Formula (Ai-): 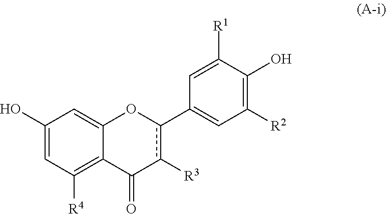
or a pharmaceutically acceptable salt thereof, wherein:
each of R¹ and R² is independently hydrogen or OR^{A};
R³ is hydrogen or OR^{A};
R⁴ is hydrogen or —OR^{A};
R^{A} is hydrogen, C₁-C₆ alkyl, or cycloalkyl; and
“ [symbol] ” is a single or double bond.

13. The composition for use of claim 12, wherein the compound of Formula (A-i) is selected from a compound of Table 1.

14. The composition for use of any one of the preceding claims, wherein the flavonoid is selected from luteolin, myricetin, taxifolin, fisetin, and kaempferol, or a pharmaceutically acceptable salt thereof.

15. The composition for use of any one of the preceding claims, wherein the flavonoid is quercetin, luteolin, or a pharmaceutically acceptable salt thereof.

16. The composition for use of any one of the preceding claims, wherein the flavonoid is luteolin or a pharmaceutically acceptable salt thereof.

17. The composition for use of any one of the preceding claims, wherein the efficacy of the combination is at least X₁-fold greater than the efficacy of the flavonoid alone at the molar amount used in the combination, wherein X₁ is 1, 1.25, 1.5, 1.75, 2, 2.5, or greater.

18. The composition for use of any one of the preceding claims, wherein the efficacy of the combination is at least X₁-fold greater than the efficacy of the xanthone alone at the molar amount used in the combination, wherein X₁ is 1, 1.25, 1.5, 1.75, 2, 2.5, or greater.

19. The composition for use of any one of the proceeding claims, wherein the xanthone is gamma-mangostin and the flavonoid is luteolin.

20. The composition for use of any one of the preceding claims, wherein the flavonoid is provided in the combination in the absence of quercetin, luteolin, myricetin, taxifolin, fisetin, or kaempferol, or a pharmaceutically acceptable salt thereof.

21. The composition for use of any one of the preceding claims, wherein the flavonoid is luteolin or a pharmaceutically acceptable salt thereof.

22. The composition for use of claim 21, wherein the luteolin or a pharmaceutically acceptable salt thereof is provided in the combination in the absence of quercetin, luteolin, myricetin, taxifolin, fisetin, or kaempferol, or a pharmaceutically acceptable salt thereof.

23. The composition for use of any one of claims 21-22, wherein the luteolin or a pharmaceutically acceptable salt thereof is provided as a pharmaceutical composition, and the pharmaceutical composition comprises less than about 90%, 95%, 99%, or 99.9% of another compound in listed in Table 1 or a pharmaceutically acceptable salt thereof.

24. The composition for use of any one of the preceding claims, wherein the xanthone is a compound selected from Table 1 or a pharmaceutically acceptable salt thereof.

25. The composition for use of claim 24, wherein the xanthone is substantially pure.

26. The composition for use of any one of the preceding claims, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of another compound.

27. The composition for use of any one of the preceding claims, wherein the xanthone is provided as a pharmaceutical composition and the pharmaceutical composition comprises less than about 500, 55%, 60%, 65%, 70%, 75%, 80%, 85%, 90%, 95%, 99%, or 99.9% of another xanthone, e.g., a xanthone listed in Table 2 or a pharmaceutically acceptable salt thereof.

28. The composition for use of any one of the preceding claims, wherein the xanthone is provided as a pharmaceutical composition and the pharmaceutical composition comprises less than about 50%, 55%, 60%, 65%, 70%, 75%, 80%, 85%, 90%, 95%, 99%, or 99.9% of a plant-derived substance.

29. The composition for use of any one of the preceding claims, wherein the flavonoid is provided in the combination in the absence of hyperforin, adhyperforin, or a pharmaceutically acceptable salt thereof.

30. The composition for use of any one of the preceding claims, wherein the xanthone is gamma-mangostin or a pharmaceutically acceptable salt thereof.

31. The composition for use of claim 30, wherein the gamma-mangostin or a pharmaceutically acceptable salt thereof is provided in the combination in the absence of alpha-mangostin, beta-mangostin, or 8-deoxygartanin, or a pharmaceutically acceptable salt thereof.

32. The composition for use of any one of the preceding claims, wherein the luteolin or a pharmaceutically acceptable salt thereof is provided as a pharmaceutical composition, and the pharmaceutical composition comprises less than about 90%, 95%, 99%, or 99.9% of another compound in listed in Table 2 or a pharmaceutically acceptable salt thereof.

33. The composition for use of any one of the preceding claims, wherein each of gamma-mangostin and luteolin is independently formulated as a pharmaceutical composition.

34. The composition for use of any one of the preceding claims, wherein the gamma-mangostin and luteolin are formulated together as a pharmaceutical composition.

35. The composition for use of any one of the preceding claims, wherein each of the gamma-mangostin and luteolin is provided (e.g., administered) concomitantly to the subject.

36. The composition for use of claim 35, wherein each of the gamma-mangostin and luteolin is provided (e.g., administered) sequentially to the subject.

37. The composition for use of claim 36, wherein the gamma-mangostin is provided (e.g., administered) to the subject prior to the luteolin.

38. The composition for use of claim 36, wherein the luteolin is provided (e.g., administered) to the subject prior to the gamma-mangostin.

39. The composition for use of any one of the preceding claims, wherein the xanthone and the flavonoid are formulated for topical, systemic, or oral administration.

40. The composition for use of any one of the preceding claims, wherein the flavonoid is administered at a dosage to provide a concentration of between 0.1 μM and 500 μM in the subject or a cell.

41. The composition for use of any one of the preceding claims, wherein the ratio of the amount of flavonoid to the xanthone in the combination is between 200:1 to 1:1.

42. The composition for use of any one of the preceding claims, wherein:
(i) the flavonoid is luteolin or a pharmaceutically acceptable salt thereof;
(ii) the xanthone is gamma-mangostin or a pharmaceutically acceptable salt thereof; and
(iii) the molar amount of the flavonoid in the combination is between 20-fold and 5-fold greater than the molar amount of the xanthone.

43. The composition for use of any one of the preceding claims, further comprising administration of an additional agent.

44. The composition for use of any one of the preceding claims, wherein the mammal is a human.
